(19) United States
(12) Patent Application Publication
(10) Pub. No.: US 2025/0288405 A1
(43) Pub. Date: Sep. 18, 2025

(54) TRANSFER INSTRUMENT

(71) Applicant: TERUMO KABUSHIKI KAISHA, Tokyo (JP)

(72) Inventor: Koichi HAYAKAWA, Ashigarakami-gun (JP)

(73) Assignee: TERUMO KABUSHIKI KAISHA, Tokyo (JP)

(21) Appl. No.: 19/224,227

(22) Filed: May 30, 2025

Related U.S. Application Data
(63) Continuation of application No. PCT/JP2023/043044, filed on Dec. 1, 2023.

(30) Foreign Application Priority Data
Dec. 2, 2022 (JP) 2022-193754

(51) Int. Cl. A61F 2/00

(52) U.S. Cl. CPC A61F 2/0063; A61F 2002/0072

(57) ABSTRACT

A transfer instrument includes: a movement regulation portion that regulates relative movement between a first shaft and a second shaft in an axial direction when a second carrier member moves between a first position where a first support portion is retracted into an outer cylinder and a second position where the first support portion is unfolded by being exposed from the outer cylinder in a distal end direction; a movement range regulation portion that defines a distal end position of a relative movement range of the first shaft; and an engagement portion that moves a first carrier member in a proximal end direction when the second carrier member moves from the second position toward the first position.

### CROSS-REFERENCES TO RELATED APPLICATIONS

This application is a continuation of International Application No. PCT/JP2023/043044 filed on Dec. 1, 2023, which claims priority to Japanese Application No. 2022-0193754 filed on Dec. 2, 2022, the entire content of both of which is incorporated herein by reference.

### TECHNOLOGICAL FIELD

The present disclosure relates to a transfer instrument for transferring a medical sheet to a treatment site of a living body.

### BACKGROUND DISCUSSION

Japanese Patent Application Publication No. 2009-000511 A discloses a transfer instrument for transferring a medical sheet (cell sheet) for use in, for example, organ transplantation to a treatment site of a living body. The transfer instrument includes a shaft and a support portion provided at a distal portion of the shaft. The support portion includes a sheet support on which the medical sheet is placed. The transfer instrument is slid while pressing an upper surface of the medical sheet placed on a support surface of the sheet support with forceps or the like to transfer the medical sheet from the support portion to the treatment site.

In the above-described related art, when the medical sheet is transferred from the support surface of the support portion to the treatment site of the living body, it is necessary to manipulate the transfer instrument while pressing the upper surface of the medical sheet with forceps or the like, which raises a concern that the medical sheet cannot be efficiently transferred to the treatment site.

### SUMMARY

(1) An aspect of the present disclosure is a transfer instrument for transferring a medical sheet to a treatment site of a living body, the transfer instrument including: an outer cylinder; a first carrier member including a first shaft disposed inside the outer cylinder to be movable along an axial direction of the outer cylinder, and a support portion having a sheet shape, disposed at a distal portion of the first shaft, and including a support surface capable of holding the medical sheet when the distal portion of the first shaft is disposed in a distal end direction with respect to a distal portion of the outer cylinder; and a second carrier member including a second shaft extending along the first shaft and provided movably along the first shaft, in which the support portion is retracted into the outer cylinder in a state of being curved and deformed at a first position where the first and second shafts are moved in a proximal end direction with respect to the outer cylinder such that the support portion is retracted into the outer cylinder, the support portion is unfolded by being exposed from the outer cylinder in the distal end direction at a second position where the first and second shafts are moved in the distal end direction with respect to the outer cylinder such that the support portion protrudes from a distal-end opening of the outer cylinder, the first shaft is shorter than the outer cylinder in the axial direction, and the first shaft is entirely located on the distal end direction side relative to a proximal-end opening of the outer cylinder at the first position, the second shaft is longer than the outer cylinder in the axial direction and protrudes from the proximal-end opening of the outer cylinder, and the transfer instrument further includes: a movement regulation portion that regulates relative movement of the first shaft and the second shaft in the axial direction between from the first position to the second position; a movement range regulation portion that defines a distal end position of a relative movement range of the first shaft in the axial direction with respect to the outer cylinder; and an engagement portion that is provided to the second carrier member and is engaged with the first carrier member to move the first carrier member in the proximal end direction when the second carrier member moves from the second position toward the first position.

Since the transfer instrument includes the second carrier member, it is possible to transfer the medical sheet from the support portion to the treatment site by using the second carrier member without using another device (forceps or the like). It is therefore possible to efficiently transfer the medical sheet to the treatment site.

Since the transfer instrument includes the movement regulation portion and the movement range regulation portion, the medical sheet can be transferred to the treatment site by a user simply pushing, in the distal end direction, the second shaft protruding from the proximal-end opening of the outer cylinder. In addition, since the transfer instrument includes the engagement portion, the first carrier member and the second carrier member can be retracted in the outer cylinder by the user simply pulling back the second shaft protruding from the proximal-end opening of the outer cylinder in the proximal end direction. Since only the second shaft out of the first shaft and the second shaft protrudes from the proximal-end opening of the outer cylinder, the user is not confused as to which of the first shaft and the second shaft should be operated. Therefore, according to this transfer instrument, the medical sheet can be efficiently transferred to the treatment site.

(2) In the transfer instrument according to (1) above, the movement range regulation portion may include a coupling member having a string shape or wire shape and connected to the outer cylinder and the first carrier member.

According to this configuration, the distal end position of the relative movement range of the first shaft can be defined with a relatively simple configuration.

(3) In the transfer instrument according to (2) above, one end of the coupling member may be connected to a proximal portion of the outer cylinder, and another end of the coupling member may be connected to a proximal portion of the first shaft.

According to this configuration, the distal end position of the relative movement range of the first shaft can be defined with a relatively simple configuration.

(4) In the transfer instrument according to (2) or (3) above, the coupling member may be a resin film.

According to this configuration, the coupling member is flexible and is excellent in durability as compared with a yarn or the like.

(5) In the transfer instrument according to any one of (1) to (4) above, the movement range regulation portion may include: a notch formed in one of the outer cylinder or the first shaft along the axial direction; and a projection that is formed on another one of the outer cylinder or the first shaft and slides along the notch.

According to this configuration, the distal end position of the relative movement range of the first shaft can be defined with a relatively simple configuration.

(6) In the transfer instrument according to any one of (1) to (5) above, the movement regulation portion may come into contact with the support portion and a distal portion of the second shaft between from the first position to the second position, to regulate relative movement in the axial direction between the first shaft and the second shaft.

According to this configuration, in a state before the support portion is unfolded in the distal end direction of the outer cylinder, relative movement in the axial direction between the first shaft and the second shaft is prevented by the movement regulation portion bringing the distal portion of the second shaft and the support portion into contact with each other.

(7) In the transfer instrument according to (6) above, the movement regulation portion may be a pressure-application portion configured by the distal portion of the second shaft, and relative displacement between the support portion and the second shaft may be restricted by the pressure-application portion pressing the support portion against an inner surface of the outer cylinder in a state where the support portion is retracted in the outer cylinder.

According to this configuration, in a state where the support portion is retracted in the outer cylinder, when the support portion is pressed against the inner surface of the outer cylinder by the pressure-application portion which is the distal portion of the second shaft, only the second shaft is suppressed from moving in the distal end direction with respect to the outer cylinder.

(8) In the transfer instrument according to (6) above, the movement regulation portion may include: a first fitting portion provided to the support portion; and a second fitting portion provided to the distal portion of the second shaft, relative displacement between the support portion and the second shaft may be restricted by fitting between the first fitting portion and the second fitting portion in a state where the support portion is retracted in the outer cylinder, and fitting between the first fitting portion and the second fitting portion may be released by the support portion being unfolded at the second position.

According to this configuration, when the first fitting portion and the second fitting portion are fitted to each other at the first position where the support portion is retracted into the outer cylinder, relative movement in the axial direction between the first shaft including the support portion and the second shaft is prevented. When fitting between the first fitting portion and the second fitting portion is released at the second position, the second shaft becomes movable in the distal end direction with respect to the support portion.

(9) In the transfer instrument according to (6) above, the movement regulation portion may include: a first engagement portion provided on a front surface of the support portion including the support surface; and a second engagement portion formed at the distal portion of the second shaft, in a state where the support portion is retracted in the outer cylinder, relative displacement between the support portion and the second shaft may be restricted by the first engagement portion and the second engagement portion facing each other in the axial direction of the second shaft and the first engagement portion and the second engagement portion being engaged with each other in the axial direction, and engagement between the first engagement portion and the second engagement portion may be released without the first engagement portion and the second engagement portion facing each other in the axial direction, by the support portion being unfolded at the second position.

According to this configuration, when the first engagement portion and the second engagement portion are engaged with each other in the axial direction at the first position where the support portion is retracted into the outer cylinder, relative movement in the axial direction between the first shaft including the support portion and the second shaft is prevented. When the engagement between the first engagement portion and the second engagement portion is released at the second position, the second shaft becomes movable in the distal end direction with respect to the support portion.

(10) In the transfer instrument according to any one of (1) to (9) above, the first shaft may have a tubular shape, the second shaft may include a shaft body inserted through the first shaft, the engagement portion may extend from a distal portion of the shaft body, and at least a part of the engagement portion may be located on the distal end direction side relative to the first shaft, and have a larger dimension in a direction perpendicular to the axial direction than an inner diameter of the first shaft.

According to this configuration, when the second carrier member moves from the second position toward the first position, a part of the engagement portion is engaged with the distal-end opening of the first shaft. When the engagement portion is engaged with the distal-end opening, the first carrier member can move relative to the outer cylinder in the proximal end direction integrally with the second carrier member.

(11) In the transfer instrument according to any one of (1) to (10) above, a second support portion having a sheet shape and including a second support surface capable of holding the medical sheet may be provided to a distal portion of the second shaft, and the second support portion may be relatively movable with respect to the support portion between a retracted position where the second support portion overlaps with the support surface and an advanced position where the second support portion is located in the distal end direction relative to the support surface.

According to this configuration, the medical sheet can be moved along a first support portion by the second support portion including the second support surface.

(12) In the transfer instrument according to (11) above, at the first position, the second support portion may be retracted inside the outer cylinder while being curved and deformed together with the support portion.

According to this configuration, even the second support portion having a width dimension can be suitably retracted inside the outer cylinder.

According to the present disclosure, the medical sheet can be transferred to the treatment site by simply pushing, in the distal end direction, the second shaft protruding from the proximal-end opening of the outer cylinder. In addition, since only the second shaft out of the first shaft and the second shaft protrudes from the proximal-end opening of the outer cylinder, the user is not confused as to which of the first shaft and the second shaft should be operated. Therefore, according to the present disclosure, the medical sheet can be efficiently transferred to the treatment site.

(13) A transfer instrument for transferring a medical sheet to a treatment site of a living body, the transfer instrument comprising: an outer cylinder; a first carrier member including a first shaft disposed inside the outer cylinder to be movable along an axial direction of the outer cylinder, and a first support portion having a sheet shape, disposed at a distal portion of the first shaft, and including a first support surface; a second carrier member including a second shaft extending along the first shaft and provided movably along the first shaft, the second carrier member including a second support portion on a distal portion of the second shaft and having a sheet shape and including a second support surface, the second support portion is relatively movable with respect to the first support portion between a retracted position where the second support portion overlaps with the first support surface and an advanced position where the second support portion is located in the distal end direction relative to the first support surface; wherein the first support portion is retracted into the outer cylinder in a state of being curved and deformed at a first position where the first and second shafts are moved in a proximal end direction with respect to the outer cylinder such that the first support portion is retracted into the outer cylinder, and the first support portion is unfolded by being exposed from the outer cylinder in the distal end direction at a second position where the first and second shafts are moved in the distal end direction with respect to the outer cylinder such that the first support portion protrudes from a distal-end opening of the outer cylinder; a movement regulation portion that regulates relative movement of the first shaft and the second shaft in the axial direction between from the first position to the second position; a movement range regulation portion that defines a distal end position of a relative movement range of the first shaft in the axial direction with respect to the outer cylinder; and an engagement portion that is provided to the second carrier member and is engaged with the first carrier member to move the first carrier member in the proximal end direction when the second carrier member moves from the second position toward the first position.

(14) A method of transferring a medical sheet to a treatment site of a living body, the method comprising: placing a transfer instrument for transferring the medical sheet to the treatment site of the living body in an initial state, the transfer instrument including an outer cylinder, a first carrier member including a first shaft disposed inside the outer cylinder to be movable along an axial direction of the outer cylinder, and a first support portion having a sheet shape, disposed at a distal portion of the first shaft, and including a first support surface, a second carrier member including a second shaft extending along the first shaft and provided movably along the first shaft, the second carrier member including a second support portion on a distal portion of the second shaft and having a sheet shape and including a second support surface, the second support portion is relatively movable with respect to the first support portion between a retracted position where the second support portion overlaps with the first support surface and an advanced position where the second support portion is located in the distal end direction relative to the first support surface, wherein the first support portion is retracted into the outer cylinder in a state of being curved and deformed at a first position where the first and second shafts are moved in a proximal end direction with respect to the outer cylinder such that the first support portion is retracted into the outer cylinder, and the first support portion is unfolded by being exposed from the outer cylinder in the distal end direction at a second position where the first and second shafts are moved in the distal end direction with respect to the outer cylinder such that the first support portion protrudes from a distal-end opening of the outer cylinder, a movement regulation portion that regulates relative movement of the first shaft and the second shaft in the axial direction between from the first position to the second position, a movement range regulation portion that defines a distal end position of a relative movement range of the first shaft in the axial direction with respect to the outer cylinder, and an engagement portion that is provided to the second carrier member and is engaged with the first carrier member to move the first carrier member in the proximal end direction when the second carrier member moves from the second position toward the first position; placing the medical sheet on the second support surface; retracting the medical sheet into the outer cylinder together with the first support portion and the second support portion to a retracted position; inserting the transfer instrument inserted into the living body and positioning a distal end of the transfer instrument near the treatment site of the living body; moving the first shaft and the second shaft in a distal end direction relative to the outer cylinder via the movement regulation portion, and unfolding the first support portion, the second support portion and the medical sheet; and moving the second carrier member in the distal end direction relative to the first carrier member so that the second support portion on which the medical sheet is placed is moved from the retracted position to an advanced position, and wherein the second support portion protrudes in the distal end direction relative to the distal end of the first support portion to move the medical sheet to above the treatment site of the living body and bringing an overhanging portion of the medical sheet into contact with the treatment site of the living body.

### BRIEF DESCRIPTION OF THE DRAWINGS

FIG. 1 is a perspective view of a transfer instrument according to a first embodiment of the present disclosure.

FIG. 2 is an exploded perspective view of the transfer instrument illustrated in FIG. 1.

FIG. 3 is a plan view of a distal portion of the transfer instrument illustrated in FIG. 1.

FIG. 4 is a longitudinal cross-sectional view taken along line IV-IV in FIG. 3.

FIG. 5 is a transverse cross-sectional view taken along line V-V in FIG. 3.

FIG. 6 is a flowchart illustrating a procedure of a transfer method for transferring a medical sheet using the transfer instrument illustrated in FIG. 1.

FIG. 7 is a first explanatory diagram of a sheet placing process.

FIG. 8 is a second explanatory diagram of the sheet placing process.

FIG. 9 is an explanatory diagram of a retracting process.

FIG. 10 is a transverse cross-sectional view taken along line X-X in FIG. 9.

FIG. 11 is a transverse cross-sectional view taken along line XI-XI in FIG. 9.

FIG. 12 is an explanatory diagram of a positioning process.

FIG. 13 is an explanatory diagram of an unfolding process.

FIG. 14 is an explanatory diagram of a moving process.

FIG. 15 is an explanatory diagram of a withdrawing process.

FIG. 16 is a cross-sectional view of a transfer instrument including a movement regulation portion according to a first modification.

FIG. 17A is a plan view of a distal portion of a transfer instrument illustrating a movement regulation portion according to a second modification, and FIG. 17B is a plan view illustrating a state where a first support portion of the transfer instrument in FIG. 17A is retracted in an outer cylinder.

FIG. 18 is a plan view of a distal portion of a transfer instrument including a first support portion according to a third modification.

FIG. 19 is a cross-sectional view taken along line XIX-XIX in FIG. 18.

FIG. 20 is an enlarged perspective view of a distal portion of a transfer instrument including a pressing body according to a fourth modification.

FIG. 21 is an enlarged perspective view of a distal portion of a transfer instrument including a pressing body according to a fifth modification.

FIG. 22 is a cross-sectional view of a distal portion of the transfer instrument of FIG. 21.

FIG. 23 is a perspective view of a transfer instrument according to a second embodiment.

FIG. 24 is a perspective view of the transfer instrument according to the second embodiment.

FIG. 25 is a perspective view of the transfer instrument according to the second embodiment.

FIG. 26 is a perspective view of the transfer instrument according to the second embodiment.

FIG. 27 is a view illustrating a modification of the transfer instrument according to the second embodiment.

### DETAILED DESCRIPTION

Set forth below with reference to the accompanying drawings is a detailed description of embodiments of a transfer instrument for transferring a medical sheet to a treatment site of a living body representing examples of the inventive transfer instrument for transferring the medical sheet to the treatment site of the living body.

### First Embodiment

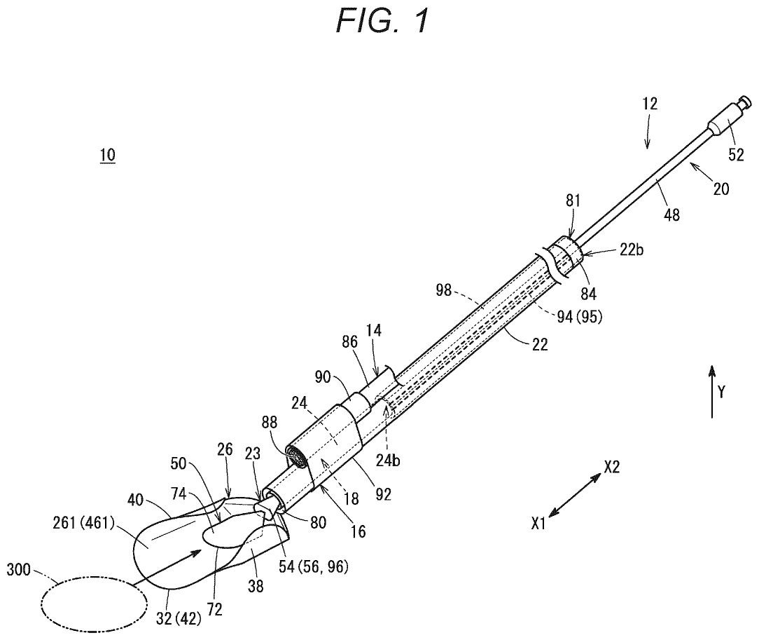

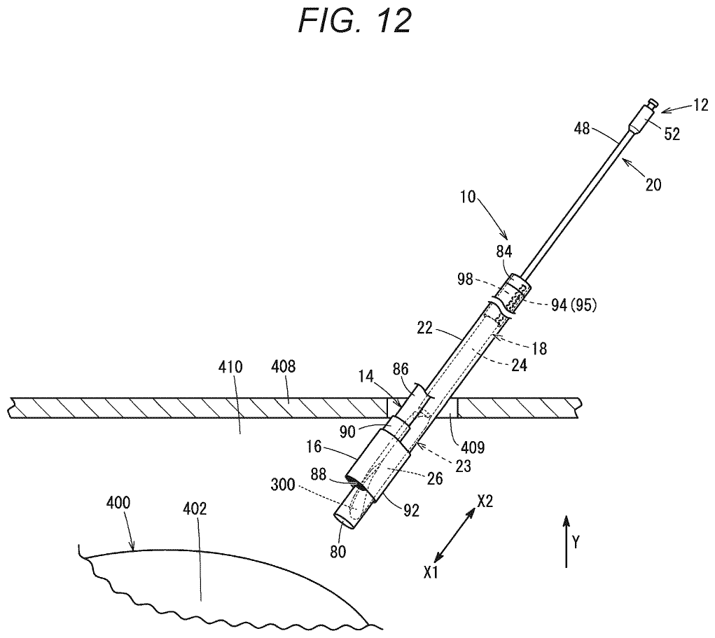

As illustrated in FIG. 1, a transfer instrument 10 according to the present embodiment is a medical instrument for transferring a medical sheet 300 to a treatment site of a living body. The transfer instrument 10 can be used for, for example, treatment of severe heart failure caused by ischemic heart disease. In this case, the medical sheet 300 is transplanted to a recipient site 402 of a heart 400 (the treatment site of the living body) (see FIGS. 12 to 15). The transfer instrument 10 is capable of attaching a plurality of the medical sheets 300 to the recipient site 402.

Examples of such a medical sheet 300 can include pharmaceutical products or regenerative medicine products for medical use, a medical instrument, and the like. The medical sheet 300 is formed in a sheet shape such as a film shape or a membrane shape (gel object). Fibrin or the like may be applied to the medical sheet 300 for reinforcement. Examples of the regenerative medicine products including cells include a cell sheet (sheet-shaped cell culture), a spheroid, and the like. It is possible to form the cell sheet by culturing autologous cells or allogenic cells. The cells constituting the cell sheet can include, for example, somatic stem cells (adult stem cells), mesenchymal stem cells, or iPS cells (induced pluripotent stem cells)-derived cardiomyocytes. Examples of the somatic stem cells preferably include skeletal myoblast cells (myoblast cells).

The medical sheet 300 may contain a tissue adhesive, a local anesthetic, or the like. The medical sheet 300 has a thickness of, for example, about 100 μm, and has a diameter of, for example, about 60 mm. Note that the thickness and the diameter (size) of the medical sheet 300 can be set as desired.

The medical sheet 300 may be a sheet to be transplanted to an organ (for example, lung, liver, pancreas, kidney, small intestine, esophagus, or the like) other than the heart 400. Further, the medical sheet 300 may be, for example, an anti-adhesion sheet as long as the sheet is for medical use.

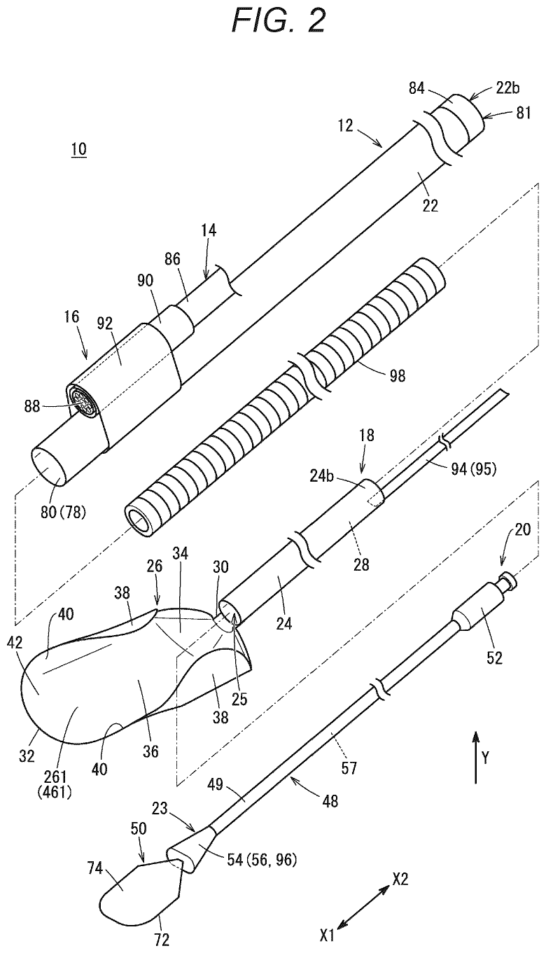

As illustrated in FIGS. 1 and 2, the transfer instrument 10 includes an instrument body 12, an endoscope 14, and a fixing member 16. The instrument body 12 includes a first carrier member 18, a second carrier member 20, an outer cylinder 22, and a movement regulation portion 23. Note that the transfer instrument 10 is not limited to a configuration including the endoscope 14.

In FIG. 2, the first carrier member 18 includes a first shaft 24 and a first support portion 26.

The first shaft 24 is a tubular body (in the present embodiment, a circular tube member) including a first lumen 28. A distal end of the first shaft 24 includes a distal-end opening 25. The first lumen 28 opens at the distal end (end in a direction of arrow X1) of the first shaft 24 via the distal-end opening 25 and opens at the proximal end (end in a direction of arrow X2) of the first shaft 24. Note that the first shaft 24 is not limited to a tubular body and may be a body other than a tubular body.

The first shaft 24 extends in an axial direction of the outer cylinder 22 and is disposed inside the outer cylinder 22 so as to be movable along the axial direction. The first shaft 24 can include, for example, a resin material. Examples of the constituent material of the first shaft 24 include, but not particularly limited to, polyethylene, polypropylene, fluororesin, polyethylene terephthalate, polymethyl methacrylate, a polyamide resin, polystyrene, polycarbonate, polyimide, polyetherimide, polyetheretherketone, polyvinyl chloride, an ABS resin, a polyamide elastomer, and a polyester elastomer. The first shaft 24 may include a metal material.

The first shaft 24 may be flexible. Note that, as illustrated in FIG. 1, the transfer instrument 10 may further include a flexible tube portion 98. The flexible tube portion 98 is fixed inside the outer cylinder 22. The flexible tube portion 98 is a member capable of maintaining a bent shape. The flexible tube portion 98 has, for example, a spiral shape (spiral structure). Note that, the flexible tube portion 98 may have a bellows shape (bellows structure) or a structure in which a plurality of annular members are connected. The flexible tube portion 98 can be made of metal, for example. The first shaft 24 is inserted through the flexible tube portion 98. In this case, the flexible tube portion 98 can bend the outer cylinder 22 into an appropriate shape and maintain the bent shape inside the outer cylinder 22.

As illustrated in FIGS. 2 to 4, the first support portion 26 is attached to the distal portion of the first shaft 24. The first support portion 26 can include, for example, a resin material. The first support portion 26 can hold the medical sheet 300. A flexible resin sheet member (film member) is bent into a predetermined form to form the first support portion 26. Alternatively, the sheet member is shaped into a predetermined form by a sheet forming die to form the first support portion 26. It is preferable that the sheet member have, but not particularly limited to, a thickness of, for example, 100 μm or more and 200 μm or less. The first support portion 26 includes a first joint 30 and a first support body 32.

The constituent material of the first support portion 26 preferably has transparency, and examples of the constituent material of the first support portion 26 can include, but not particularly limited to, polyethylene, polycarbonate, polyamide, polystyrene, polypropylene, a polyacetal resin, polyimide, polyetherimide, polyetheretherketone, polyethylene terephthalate, and fluororesin. Further, the first support portion 26 may have a mesh shape.

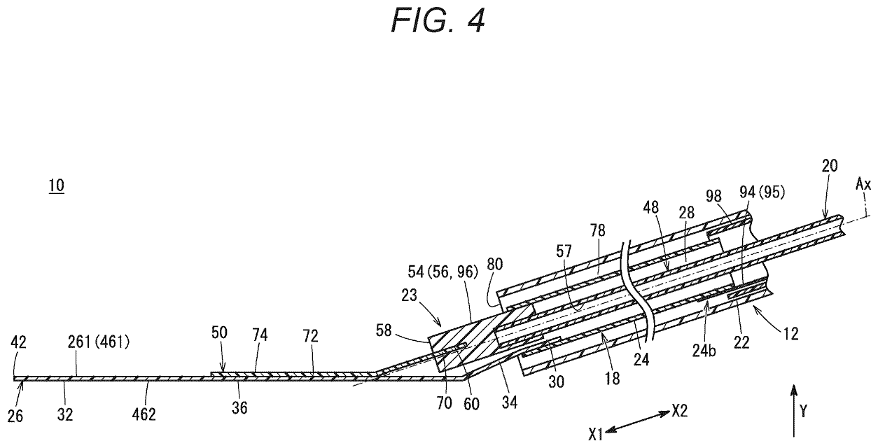

In FIG. 4, the first joint 30 is bonded to an inner peripheral surface of the distal portion of the first shaft 24 with an adhesive. Examples of the adhesive include, but not particularly limited to, a UV adhesive, a hot-melt adhesive, and an instant adhesive (for example, cyanoacrylate-based instant adhesive). The first joint 30 may be thermally fused to the inner peripheral surface of the first shaft 24.

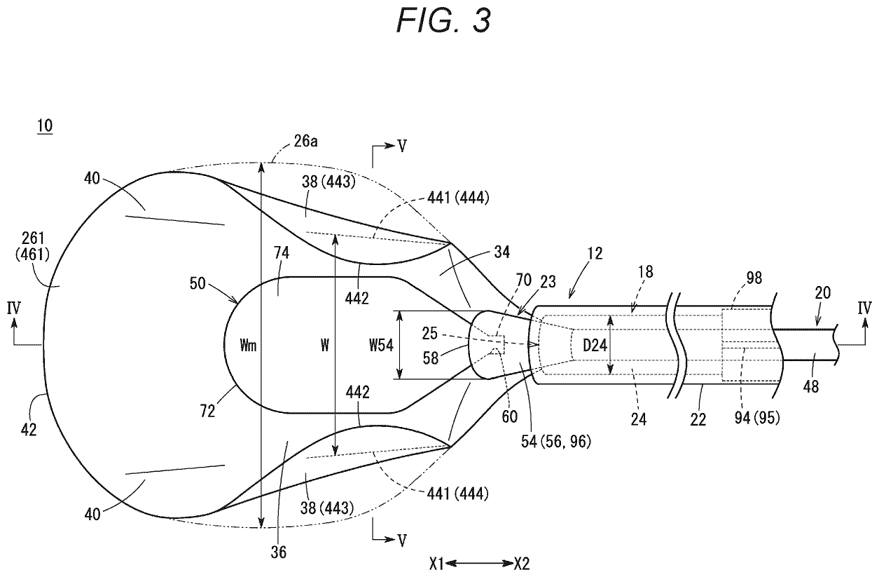

As illustrated in FIG. 3, the first support body 32 extends in a distal end direction from the first joint 30 (see FIG. 4). The first support body 32 includes a proximal-end support portion 34, an intermediate support portion 36, a pair of first protrusions 38, a pair of second protrusions 40, and a distal-end support portion 42.

The proximal-end support portion 34 extends roughly along an axis Ax of the first shaft 24 from a distal end of the first joint 30 in the distal end direction (the direction of arrow X1) (see FIG. 4). The proximal-end support portion 34 is formed to be wider in its extending direction. In other words, both sides of the proximal-end support portion 34 in a width direction are tapered toward the first joint 30.

The intermediate support portion 36 intersects with the axis Ax of the first shaft 24 in a distal end direction (in the direction of arrow X1), and extends from a distal end of the proximal-end support portion 34 in the distal end direction (in the direction of arrow X1) of the first support portion 26 (see FIG. 4). The intermediate support portion 36 is formed in a tapered shape so as to be gradually narrower in width from the distal end toward the proximal end (in the direction of arrow X2).

In FIGS. 2 and 3, the pair of first protrusions 38 protrude upward (in the direction of arrow Y) from both sides of the intermediate support portion 36 in the width direction orthogonal to a direction in which the first shaft 24 moves and inward in the width direction of the intermediate support portion 36. The pair of first protrusions 38 are connected to the proximal-end support portion 34.

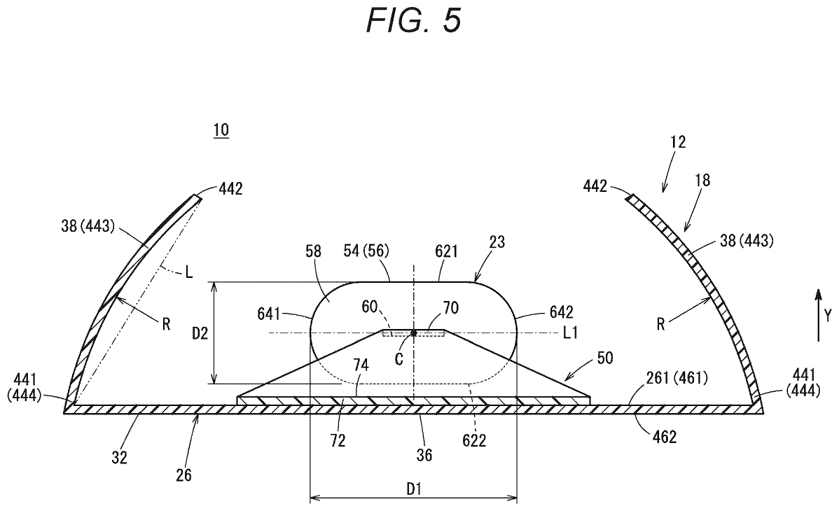

As illustrated in FIG. 5, each of the pair of first protrusions 38 includes a fixed end 441 connected to an upper surface (first support surface 261) of the intermediate support portion 36 and a free end 442 that is an end portion separated from the first support surface 261 in a protruding direction of the first protrusion 38.

In a cross section orthogonal to the protruding direction of the first protrusion 38 illustrated in FIG. 5 and orthogonal to the first support surface 261, each cross section of the first protrusions 38 includes an intermediate portion 443 constituting a portion between the free end 442 and the fixed end 441. The intermediate portion 443 has an arc shape bulging in a convex shape in a direction away from the first support surface 261. A cross section of the intermediate portion 443 is not limited to an arc shape having a single radius, and is only required to be an arc shape. In other words, in the cross section of the first protrusion 38 illustrated in FIG. 5, the intermediate portion 443 is disposed outward with respect to a line segment L connecting the fixed end 441 and the free end 442.

In a cross section orthogonal to the protruding direction of the first protrusion 38 illustrated in FIG. 5 and orthogonal to the first support surface 261, a curvature R of the intermediate portion 443 increases in a proximal end direction (the direction of arrow X2 in FIG. 3) of the first support portion 26.

As illustrated in FIG. 3, the pair of second protrusions 40 are connected to distal ends of the pair of first protrusions 38. The pair of second protrusions 40 protrude upward from both sides of the intermediate support portion 36 in the width direction and outward in the width direction of the intermediate support portion 36. Each second protrusion 40 is formed with a smaller curvature than the first protrusion 38. The second protrusions 40 are lower in protrusion height relative to the first support surface 261 than the first protrusions 38.

The distal-end support portion 42 is connected to a distal end of the intermediate support portion 36 and distal ends of the pair of second protrusions 40. The distal-end support portion 42 protrudes in an arc shape in the distal end direction (the direction of arrow X1). That is, when viewed from a direction orthogonal to the first support surface 261 illustrated in FIG. 3, the distal-end support portion 42 of the first support portion 26 has an arc shape connecting the pair of second protrusions 40.

Each of the pair of first protrusions 38 includes a pair of bent portions 444 on a proximal end side of the fixed end 441. Each of the pair of bent portions 444 causes a corresponding one of the pair of first protrusions 38 to bend from the first support surface 261 (intermediate support portion 36) of the first support portion 26 (see FIG. 5). As illustrated in FIG. 3, when the bent portion 444 of the first support portion 26 is bent to form the pair of first protrusions 38 bulging outward in a convex shape, it is preferable to bend both end portions at the bent portion 444 in a portion having a maximum width Wm in a width direction of a resin sheet member 26a (see an imaginary line shape in FIG. 3) that is to be the first support portion 26.

When viewed from a direction orthogonal to the first support surface 261 illustrated in FIG. 3, an interval W in a width direction of the pair of bent portions 444 in the first support portion 26 gradually decreases in the proximal end direction (the direction of arrow X2). That is, in the first support body 32, the pair of bent portions 444 are formed in a tapered shape in the proximal end direction.

The first support body 32 includes a front surface 461 that faces upward (in the direction of arrow Y) and includes the first support surface 261, and a back surface 462 that is a surface opposite to the front surface 461. The first support surface 261 includes a flat surface continuous over an upper surface of the proximal-end support portion 34 and upper surfaces of the intermediate support portion 36 and the distal-end support portion 42. A lubricant may be applied to the first support surface 261 so as to allow a second support portion 50 (to be described later) of the second carrier member 20 to smoothly slide on the first support surface 261.

As illustrated in FIG. 2, the second carrier member 20 includes a second shaft 48, the second support portion 50, and a hub 52. Note that, instead of providing the hub 52, a handle that can be gripped by the user may be provided at a proximal end of the second shaft 48.

The second shaft 48 is a tubular body (in the present embodiment, a circular tube member) including a second lumen 57. The second shaft 48 is longer in the axial direction than the first shaft 24. The second shaft 48 is inserted through the first lumen 28 of the first shaft 24 (see FIGS. 1 and 4). In other words, a distal portion of the second shaft 48 protrudes in the distal end direction (the direction of arrow X1) from the distal-end opening 25 of the first shaft 24. A proximal portion of the second shaft 48 protrudes in the proximal end direction (the direction of arrow X2) from the proximal-end opening of the first shaft 24 (see FIG. 1). The second shaft 48 is provided extending along the first shaft 24 so as to be movable along the first shaft 24. Note that the second shaft 48 is not limited to a tubular body and may be a body other than a tubular body.

Note that a resilient member such as a spring may be provided between the first lumen 28 of the first shaft 24 and the second shaft 48, and the second shaft 48 may be biased in the proximal end direction (the direction of arrow X2) with respect to the first shaft 24 by a resilient force of the resilient member. As a result, when the user moves the second shaft 48 in the distal end direction (the direction of arrow X1) with respect to the first shaft 24, the user's operation force disappears, so that the second shaft 48 is movable in the proximal end direction by the resilient force.

The second shaft 48 is configured to follow the shape of the first support portion 26. As the constituent material of the second shaft 48, for example, a material more flexible than the constituent material of the first shaft 24 is selected. Specifically, examples of the constituent material of the second shaft 48 include a polyamide elastomer, a polyester elastomer, a polyurethane elastomer, polyvinyl chloride, polybutadiene, a silicone rubber, and a metal coil (including a composite with a resin). The second shaft 48 is flexible.

As illustrated in FIG. 4, the second shaft 48 includes a carrier holding portion 54 and a pressure-application portion 56 that is the distal portion of the second shaft 48. The pressure-application portion 56 includes an elastic body such as an elastomer member. The pressure-application portion 56 presses the first support portion 26 against an inner surface of the outer cylinder 22 with the first support portion 26 retracted in the outer cylinder 22.

A distal end of the carrier holding portion 54 includes a pressing surface 58. The carrier holding portion 54 can cause the pressing surface 58 to press an outer edge surface of the medical sheet 300 supported by the first support portion 26 in the distal end direction (the direction of arrow X1). In the present embodiment, the pressure-application portion 56 is provided with the carrier holding portion 54 that supports the second support portion 50. The carrier holding portion 54 includes the pressing surface 58 and an attachment hole 60. The carrier holding portion 54 is formed to be wider in the distal end direction when viewed from above. The carrier holding portion 54 is formed in a trapezoidal shape when viewed from above. In a cross section orthogonal to an axis of the second shaft 48 illustrated in FIG. 5, the carrier holding portion 54 has a line-symmetrical shape with respect to an imaginary line L1 passing through a center line C of the carrier holding portion 54 and parallel to a width direction of the carrier holding portion 54. In other words, the carrier holding portion 54 has a symmetrical shape in a vertical direction with respect to the imaginary line L1.

Specifically, in a cross section orthogonal to the axis of the second shaft 48, a cross section of the carrier holding portion 54 includes a pair of linear portions 621, 622 orthogonal to the axis of the second shaft 48 and a pair of convex-shaped portions 641, 642 that are disposed on both sides of the pair of linear portions 621, 622 and are convex in a direction (outward in the width direction) away from the linear portions 621, 622. The convex-shaped portions 641, 642 are formed to have an arc cross-sectional shape. A first length D1 (width dimension) of the carrier holding portion 54 along an extending direction of the linear portions 621, 622 is larger than a second length D2 (thickness dimension) of the carrier holding portion 54 orthogonal to the extending direction. That is, the carrier holding portion 54 has a flattened cross-sectional shape. Note that the carrier holding portion 54 is not limited to a configuration formed in a flattened cross-sectional shape, and may be formed in, for example, a circular cross-sectional shape, an elliptical cross-sectional shape, a square cross-sectional shape, a rectangular cross-sectional shape, or the like. In addition, instead of the convex-shaped portions 641, 642, the carrier holding portion 54 may have an uneven structure in which the first support portion 26 is pressed against the inner surface of the outer cylinder 22 in a state where the first support portion 26 is retracted in the outer cylinder 22.

When the first support portion 26 is retracted in a lumen 78 of the outer cylinder 22 together with the pressure-application portion 56, the width (first length D1) of the pressure-application portion 56 is set such that the first support portion 26 is pushed toward the lumen 78 of the outer cylinder 22 by both end portions (pair of convex-shaped portions 641, 642) of the pressure-application portion 56 in the width direction. The first length D1 of the pressure-application portion 56 is set to be equal to or slightly larger than an inner diameter of the outer cylinder 22 (a diameter of the lumen 78), for example. Since the first support portion 26 has a thickness, the first length D1 of the pressure-application portion 56 may be set slightly smaller than the inner diameter of the outer cylinder 22.

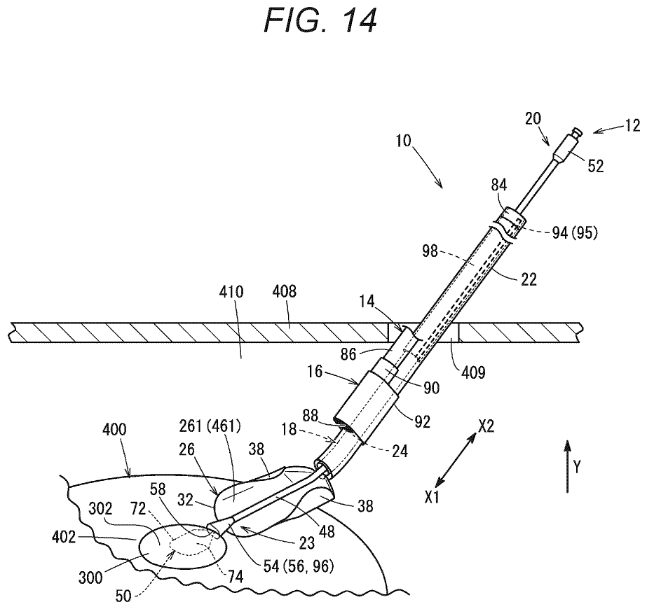

In FIG. 4, the pressing surface 58 is provided on a distal end surface of the carrier holding portion 54. The attachment hole 60 opens at the pressing surface 58. The second support portion 50 is attached to the pressing surface 58. The pressing surface 58 presses the outer edge surface of the medical sheet 300 in the distal end direction (the direction of arrow X1) (see FIG. 14). The pressing surface 58 is a flat surface orthogonal to an axis of the carrier holding portion 54. Note that, the pressing surface 58 may include a supply hole through which a liquid for priming (for example, a saline solution) can be supplied toward the distal portion of the second shaft 48.

The attachment hole 60 opens in the pressing surface 58 of the carrier holding portion 54. The attachment hole 60 is disposed on the center line C of the carrier holding portion 54 on the pressing surface 58. The attachment hole 60 has a slit shape extending parallel to the width direction of the carrier holding portion 54 from the center line C. The attachment hole 60 has a symmetrical shape in the width direction with respect to the center line C. The attachment hole 60 extends in the axial direction from the pressing surface 58. A part of the second support portion 50 is inserted into and connected to the attachment hole 60.

In FIGS. 2 to 4, the second support portion 50 is a flexible sheet. The second support portion 50 includes a second joint 70 and a second support body 72. The second joint 70 is provided at a proximal end of the second support portion 50. The second joint 70 is provided at a proximal end of the second support body 72. The second joint 70 is inserted into and, for example, bonded to the attachment hole 60 of the carrier holding portion 54. The second joint 70 may be joined to the attachment hole 60 of the carrier holding portion 54 by a suitable joining method other than adhesion. Further, the second support portion 50 may be integrally formed with the carrier holding portion 54.

The second support body 72 extends in the distal end direction (the direction of arrow X1) from the second joint 70. The second support body 72 extending from the second joint 70 is shorter in the extending direction than the first support body 32 extending from the first joint 30. The second support body 72 is provided with, on its upper surface, a second support surface 74 on which the medical sheet 300 is placed. The second support surface 74 includes a flat surface. The second support body 72 is smaller than the first support body 32. That is, the second support surface 74 is smaller in area than the first support surface 261.

As illustrated in FIG. 3, a proximal portion of the second support body 72 is formed to be narrower in the proximal end direction (the direction of arrow X2). The intermediate portion 443 between the distal end and the proximal end of the second support body 72 extends with a substantially constant width. The distal portion of the second support portion 50 protrudes in an arc shape toward the distal end direction (the direction of arrow X1). A lubricant may be applied to both surfaces (lower surface and upper surface) of the second support body 72.

In FIG. 2, the hub 52 is attached to the proximal portion of the second shaft 48.

In FIGS. 1 and 2, the outer cylinder 22 is a cylindrical member including the lumen 78. The lumen 78 includes a distal-end opening 80 that opens at a distal end (end in the direction of arrow X1) of the outer cylinder 22. The lumen 78 opens at a proximal end (end in the direction of arrow X2) of the outer cylinder 22. The outer cylinder 22 is flexible. Examples of the constituent material of the outer cylinder 22 are the same as the examples of the constituent material of the first shaft 24 described above.

The first shaft 24 is inserted through the lumen 78 of the outer cylinder 22. The outer cylinder 22 is shorter in the axial direction than the first shaft 24. In FIGS. 3 and 4, an inner diameter of the outer cylinder 22 is smaller than a width of the intermediate support portion 36. The width of the intermediate support portion 36 is substantially identical to a circumferential length of the inner surface of the outer cylinder 22 or less than the circumferential length of the inner surface of the outer cylinder 22 so as to allow the first support portion 26 to be retracted into the outer cylinder 22 with the first support portion 26 curled into a cylinder along a circumferential direction of an inner peripheral surface of the outer cylinder 22. The outer cylinder 22 is provided with, at a proximal end of the outer cylinder 22, an airtight valve 84 that is in contact or relatively close contact with an outer peripheral surface of the second shaft 48.

In FIGS. 2 and 4, a distal end surface of the outer cylinder 22 extends orthogonal to the axial direction of the outer cylinder 22.

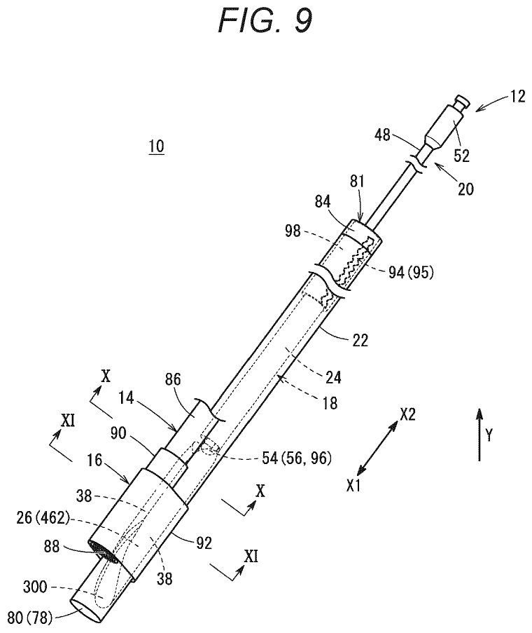

Note that, in an axial direction of the outer cylinder 22, the first shaft 24 is shorter than the outer cylinder 22. In a state where the first support portion 26 is retracted into the outer cylinder 22, the entire first shaft 24 is located on the distal end direction side relative to a proximal-end opening 81 of the outer cylinder 22 (see also FIG. 9). That is, in a state where the first support portion 26 is retracted into the outer cylinder 22, the first shaft 24 does not protrude from the proximal-end opening 81 of the outer cylinder 22 in the proximal end direction. Therefore, even in a case where the first support portion 26 protrudes from the distal-end opening 80 of the outer cylinder 22, the first shaft 24 does not protrude from the proximal-end opening 81 of the outer cylinder 22 in the proximal end direction.

Further, in the present embodiment, the second shaft 48 is inserted through the first lumen 28 of the first shaft 24. In the axial direction of the outer cylinder 22, the second shaft 48 is longer than the outer cylinder 22. Therefore, the second shaft 48 protrudes in the proximal end direction from the proximal-end opening 81 of the outer cylinder 22.

As illustrated in FIG. 2, the endoscope 14 includes an elongated endoscope main body 86. A distal portion of the endoscope main body 86 is fixed to an outer peripheral surface of the outer cylinder 22 by the fixing member 16 (see FIG. 1). An objective lens 88 provided on a distal end surface of the endoscope main body 86 is oriented the distal end direction of the outer cylinder 22 (in the direction of arrow X1). The distal portion of the endoscope main body 86 is fixed to an intermediate portion of the outer cylinder 22 in the axial direction. Note that the distal portion of the endoscope main body 86 may be fixed to a distal portion of the outer cylinder 22.

The fixing member 16 can include, for example, a fixing cylinder 90 and a fixing tube 92. The fixing cylinder 90 can include, for example, a hard resin material. The endoscope main body 86 can be inserted into a lumen of the fixing cylinder 90. The fixing cylinder 90 is disposed along a longitudinal direction of the outer cylinder 22. The fixing tube 92 is a tube for fixing the fixing cylinder 90 at a predetermined position of the outer cylinder 22. The fixing tube 92 can be, for example, a heat-shrink tube. Note that the outer cylinder 22 and the fixing cylinder 90 may be integrally molded. Note that how to fix the distal portion of the endoscope main body 86 to the outer cylinder 22 may be determined as desired.

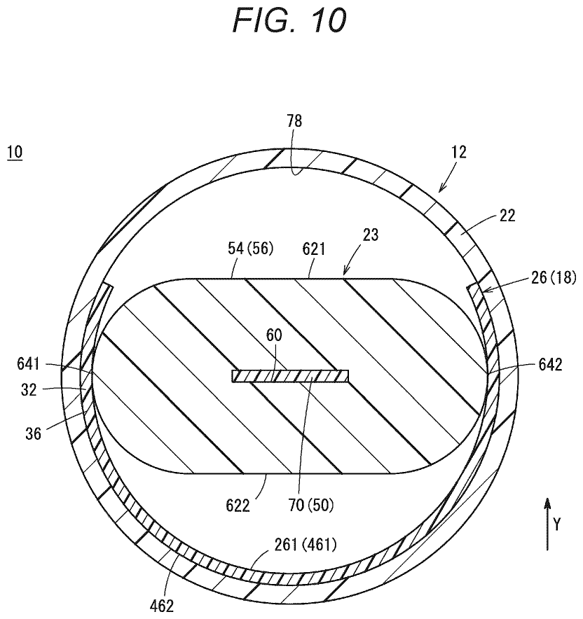

The movement regulation portion 23 can restrict relative movement between the first shaft 24 and the second shaft 48 in the axial direction. The movement regulation portion 23 is configured by the carrier holding portion 54 (pressure-application portion 56) configured by a distal portion of the second shaft 48. By the carrier holding portion 54 pressing the first support portion 26 against the inner surface (lumen 78) of the outer cylinder 22 in a state where the first support portion 26 is retracted in the outer cylinder 22, relative displacement between the first support portion 26 and the second shaft 48 is restricted. As the relative movement between the first support portion 26 and the second shaft 48 is restricted, relative movement between the first shaft 24 and the second shaft 48 in the axial direction is restricted (see FIG. 10).

The transfer instrument 10 further includes a movement range regulation portion 94 and a third engagement portion (engagement portion) 96.

The movement range regulation portion 94 defines a distal end position of a relative movement range of the first shaft 24 with respect to the outer cylinder 22. The movement range regulation portion 94 of the present embodiment is configured by a coupling member 95.

The coupling member 95 is a member having a string shape or a wire shape. The coupling member 95 is connected to the outer cylinder 22 and the first carrier member 18. In the present embodiment, a case will be described in which one end of the coupling member 95 is fixed to a proximal portion 22b of the outer cylinder 22 and another end of the coupling member 95 is fixed to a proximal portion 24b of the first shaft 24 of the first carrier member 18. More specifically, one end of the coupling member 95 is bonded to an inner surface of the proximal portion 22b of the outer cylinder 22. Further, another end of the coupling member 95 is bonded to the proximal portion 24b of the first shaft 24. Note that, another end of the coupling member 95 is connected to an outer surface of the first shaft 24 (see also FIG. 4), but may be connected to an inner surface of the first shaft 24.

The coupling member 95 is flexible. Therefore, when the first shaft 24 moves relative to the outer cylinder 22 in the proximal end direction, the coupling member 95 can be bent. As a result, the first shaft 24 and the outer cylinder 22 are allowed to relatively move such that the proximal portion 24b of the first shaft 24 and the proximal portion 22b of the outer cylinder 22 approach each other.

On the other hand, when the first shaft 24 moves relative to the outer cylinder 22 in the distal end direction, the coupling member 95 is stretched gradually as a distance between the proximal portion 22b of the outer cylinder 22 and the proximal portion 24b of the first shaft 24 increases. The stretched coupling member 95 prevents the distance between the proximal portion 22b of the outer cylinder 22 and the proximal portion 24b of the first shaft 24 from becoming larger than an axial length of the coupling member 95. In this manner, the coupling member 95 defines the distal end position of the relative movement range of the first shaft 24. That is, a position of the first shaft 24 when the coupling member 95 is stretched to the maximum is defined as the distal end position of the relative movement range of the first shaft 24.

Note that, such a coupling member 95 can be achieved by, for example, a hard resin film. The hard resin as the material of the film can be, for example, polyimide, but is not limited to a polyimide. The hard resin film is flexible and is excellent in durability as compared with a yarn or the like. In addition, the hard resin film is less likely to stretch than rubber, an elastomer material, or the like. Therefore, the hard resin film can more accurately define the distal end position of the relative movement range of the first shaft 24 with respect to the outer cylinder 22, as compared with rubber, an elastomer material, or the like. The coupling member 95 may be connected to a portion of the first shaft 24 other than the proximal portion 24b. Furthermore, the coupling member 95 may be connected to a portion of the outer cylinder 22 other than the proximal portion 22b.

The third engagement portion 96 is provided to the second carrier member 20. The third engagement portion 96 is engaged with the first carrier member 18 when the second carrier member 20 moves from a second position to a first position described later. The first carrier member 18 can move relative to the outer cylinder 22 in the proximal end direction integrally with the second carrier member 20 by being engaged with the third engagement portion 96.

In the following description, a portion of the second shaft 48 on the proximal end direction side (the direction of arrow X2) relative to the third engagement portion 96 is also referred to as a shaft body 49. That is, the second shaft 48 includes the shaft body 49 (see also FIG. 2). The shaft body 49 is inserted through the first shaft 24. The third engagement portion 96 extends in a distal end direction from a distal portion of the shaft body 49.

At least a part of the third engagement portion 96 is located on the distal end direction side relative to the first shaft 24 (see also FIG. 3). In the third engagement portion 96, a width of a portion located on the distal end direction side relative to the first shaft 24 is larger than a diameter of the first lumen 28. That is, in a radial direction of the first shaft 24, a dimension of at least a part of the third engagement portion 96 is larger than an inner diameter D24 of the first shaft 24. The radial direction of the first shaft 24 is perpendicular to the axial direction of the outer cylinder 22.

In the present embodiment, the carrier holding portion 54 described above also serves as the third engagement portion 96. That is, as illustrated in FIG. 3, at least a part of the carrier holding portion 54 is located on the distal end direction side relative to the first shaft 24. In the carrier holding portion 54, a width W54 of a portion located on the distal end direction side relative to the first shaft 24 is larger than the inner diameter D24 of the first shaft 24 (W54>D24). When the second carrier member 20 moves from the second position to the first position, a portion of the carrier holding portion 54 having the width W54 is engaged with the distal-end opening 25 of the first shaft 24. By the carrier holding portion 54 being engaged with the distal-end opening 25 of the first shaft 24, the entire first carrier member 18 can relatively move in the proximal end direction together with the second carrier member 20.

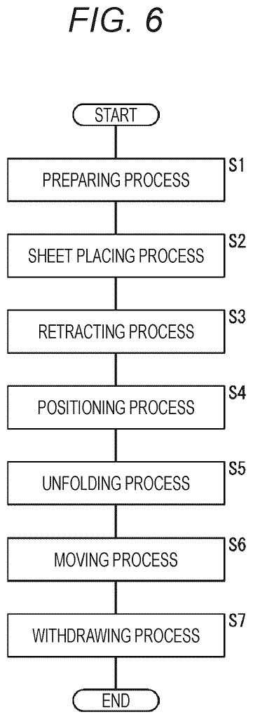

Next, a transfer method for transferring the medical sheet 300 to a treatment site of a living body will be described. Specifically, as illustrated in FIGS. 12 to 15, the transfer method for transferring the medical sheet 300 to the recipient site 402 of the heart 400 (the treatment site of the living body) during thoracoscopic surgery will be described. As illustrated in FIG. 6, the transfer method according to the present embodiment includes a preparing process, a sheet placing process, a retracting process, a positioning process, an unfolding process, a moving process, and a withdrawing process.

First, in the preparing process (S1), the transfer instrument 10 according to the present embodiment described above is prepared. The following description will be given on the assumption that a state as illustrated in FIG. 1 is an initial state of the transfer instrument 10. In the initial state, the first and second shafts 24 and 48 are moved in the distal end direction (the direction of arrow X1) relative to the outer cylinder 22 so as to be at a protruding position (second position) where the first support portion 26 and the second support portion 50 protrude from the distal-end opening 80 of the outer cylinder 22 in the distal end direction. The first and second support portions 26 and 50 are each unfolded when moving out of the outer cylinder 22 in the distal end direction, and the second support portion 50 is disposed on the first support surface 261 of the first support portion 26. That is, the second support portion 50 is disposed at a retracted position where the second support portion 50 is placed on the first support surface 261 of the first support portion 26. At this time, a proximal portion of the carrier holding portion 54 is in the first lumen 28 of the first shaft 24.

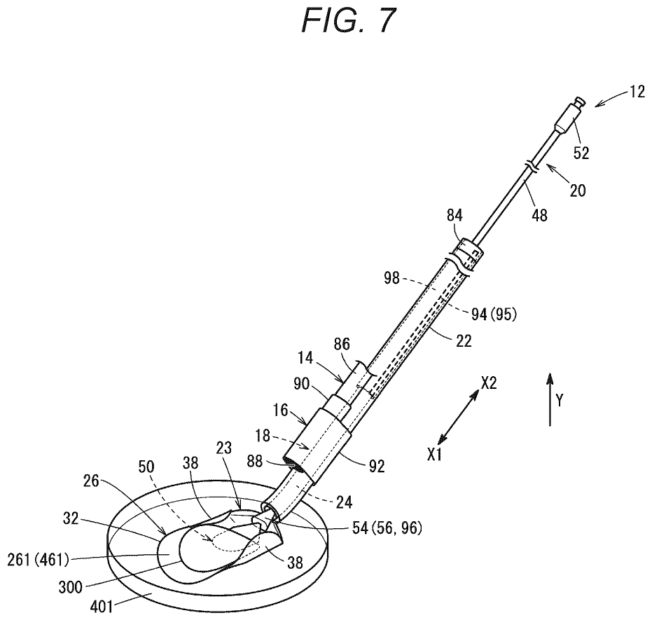

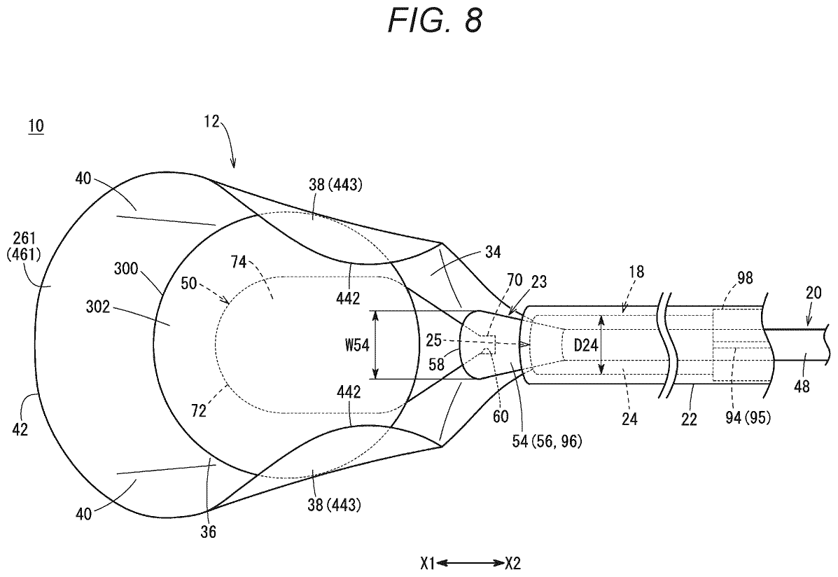

Next, in the sheet placing process (S2), as illustrated in FIG. 7, the medical sheet 300 placed on a Petri dish 401 is placed on the second support surface 74. Note that, as illustrated in FIG. 8, the medical sheet 300 sticks out over the second support portion 50 with the medical sheet 300 placed on the second support surface 74. The first support surface 261 supports an overhanging portion 302 of the medical sheet 300 that sticks out from the second support portion 50. The pair of second protrusions 40 prevent the medical sheet 300 from moving (becoming misaligned) in the width direction of the intermediate support portion 36 with the medical sheet 300 placed on the second support surface 74.

Subsequently, in the retracting process (S3 in FIG. 6), the medical sheet 300 is retracted into the outer cylinder 22 together with the first support portion 26 and the second support portion 50 at a retracted position (first position). Specifically, the user holds and pulls the second shaft 48 in the proximal end direction. Accordingly, the third engagement portion 96 can be engaged with the first shaft 24. By further pulling the second shaft 48 in the proximal end direction in a state where the third engagement portion 96 is engaged with the first shaft 24, the first shaft 24 can be moved in the proximal end direction as the second shaft 48 moves in the proximal end direction.

Accordingly, the proximal-end support portion 34 is pulled in the proximal end direction through the distal-end opening 80 of the outer cylinder 22. At this time, when both the tapered sides of the proximal-end support portion 34 come into contact with the distal-end opening 80 of the outer cylinder 22, a force acts on the proximal-end support portion 34 to curl along the circumferential direction of the outer cylinder 22. Therefore, the proximal-end support portion 34 is smoothly pulled into the outer cylinder 22 while curling. At this time, the first support portion 26 is retracted into the outer cylinder 22 while curling in a conical shape such that the distal end side of the first support portion 26 becomes larger in diameter than the proximal-end support portion 34.

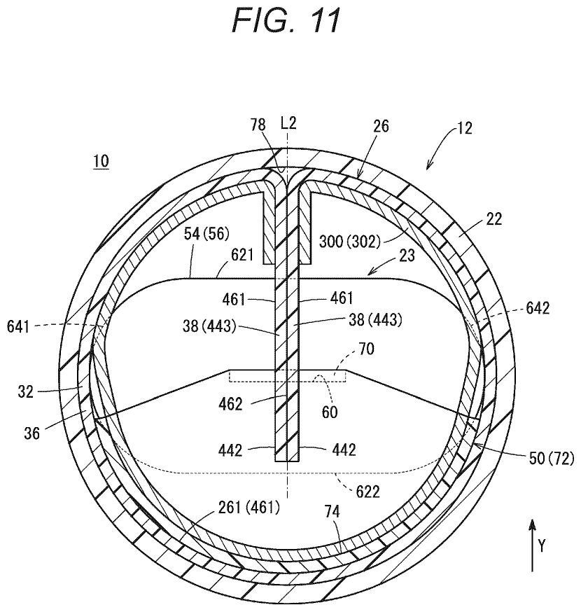

When the proximal-end support portion 34 becomes deformed, a force acts on the intermediate support portion 36 to cause the intermediate support portion 36 to curl along the circumferential direction of the outer cylinder 22, so that the intermediate support portion 36 is pulled into the outer cylinder 22 while curling. At this time, the intermediate support portion 36 becomes deformed into a cylindrical shape along the inner surface of the outer cylinder 22. Each of the pair of first protrusions 38 is curved such that its fixed end 441 is rolled inward, and the front surface 461 of the first support portion 26 faces inward, and the back surface 462 of the first support portion 26 faces outward. As illustrated in FIG. 11, the back surfaces 462 of the intermediate portions 443 bulging in a convex shape radially outward come into contact with each other on an imaginary line L2 extending in a direction orthogonal to a center axis of the outer cylinder 22. The intermediate portion 443 of one of the first protrusions 38 and the intermediate portion 443 of another first protrusion 38 come into contact with each other and are retracted downward (toward the first support surface 261, the front surface 461).

Accordingly, the back surface 462 of the first support portion 26 is curved to come into contact or relatively close contact with the inner surface of the outer cylinder 22, each of the first protrusions 38 is further curved from the fixed end 441 toward the free end 442 to fold back toward the center of the outer cylinder 22, and the pair of free ends 442 are disposed below the center axis of the outer cylinder 22. That is, the first support portion 26 is curved in a heart shape along the inner surface of the outer cylinder 22.

The heart shape refers to a roughly round shape including two convex shapes, i.e., a convex shape on one side and a convex shape on the other side. In a case where the heart shape is formed in the lumen 78 of the tubular body (outer cylinder 22), the two convex shapes protruding toward the opposite sides along the inner surface of the tubular body come close to each other so as to bring their respective peripheral surfaces into partial contact with each other, so that the entire contour becomes a roughly round shape along the inner surface of the tubular body (refer to the shape of the first support portion 26 in FIG. 11).

Along with the curved deformation of the first support portion 26, the second support portion 50 similarly undergoes curved deformation along the first support portion 26 inside the first support portion 26 (on the front surface 461 side). Along with the curved deformation of the first support portion 26 and the second support portion 50, the medical sheet 300 is deformed into a shape corresponding to the shape of the first support body 32 and the shape of the second support body 72, and the medical sheet 300 is retracted into the outer cylinder 22 accordingly. As illustrated in FIG. 9, when the first support portion 26 is entirely inserted into the outer cylinder 22, the retracting process is complete. Note that, as the first shaft 24 moves in the proximal end direction, the coupling member 95 described above can be bent. In a state where the retracting process is completed, the coupling member 95 maintains a bent state (see also FIG. 9).

In the retracted state where the first support portion 26, the second support portion 50, and the medical sheet 300 are retracted into the outer cylinder 22, as illustrated in FIG. 10, the pair of first protrusions 38 are located adjacent to the distal end (in the direction of arrow X1) relative to the pressing surface 58 of the pressure-application portion 56 with the back surfaces 462 in contact with each other (see FIG. 9).

Subsequently, in the positioning process (S4 in FIG. 6), as illustrated in FIG. 12, the transfer instrument 10 is inserted into a chest cavity 410 through an incision 409 of a chest 408. At this time, the distal end of the transfer instrument 10 is positioned near the recipient site 402 of the heart 400, and the distal end of the endoscope 14 is positioned in the chest cavity 410. Note that before the transfer instrument 10 is inserted into the chest cavity 410, a liquid supply instrument may be connected to a connection port of the hub 52 to introduce a liquid (for example, a saline solution).

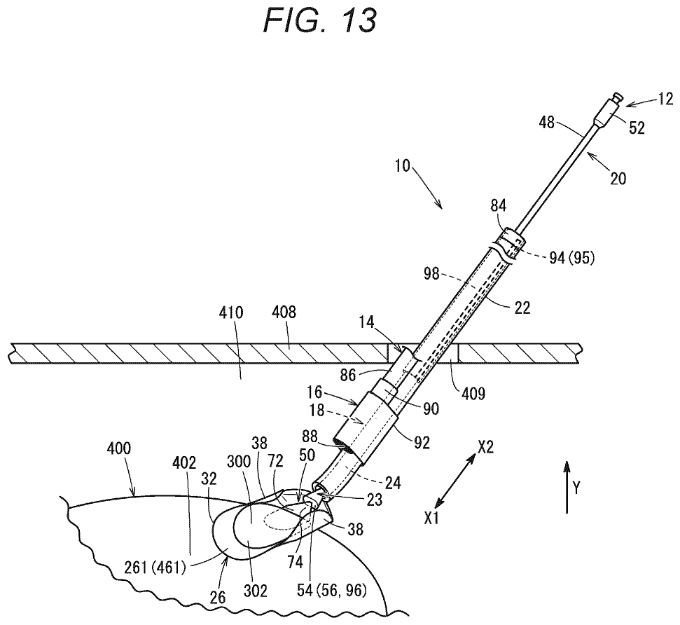

Subsequently, in the unfolding process (S5 in FIG. 6), as illustrated in FIG. 13, the first support portion 26, the second support portion 50, and the medical sheet 300 are unfolded. Specifically, in the unfolding process, with the second shaft 48 gripped, the second shaft 48 is moved in the distal end direction (the direction of arrow X1) relative to the outer cylinder 22. At this time, the first shaft 24 and the second shaft 48 integrally move in the distal end direction via the movement regulation portion 23. Then, the first support portion 26 that has moved out of the distal-end opening 80 of the outer cylinder 22 returns to the original shape due to its restoring force. At the second position where the first support portion 26 is unfolded, the second support portion 50 and the medical sheet 300 unroll flat.

In the unfolding process, of the second carrier member 20, the second support surface 74 on which the medical sheet 300 is placed is entirely located on the first support surface 261. At this time, the medical sheet 300 is supported by the first support surface 261 and the second support surface 74. It is therefore possible to prevent the overhanging portion 302 of the medical sheet 300 from becoming crinkled before the medical sheet 300 is transferred to the recipient site 402 of the heart 400.

Next, in the moving process (S6 in FIG. 6), as illustrated in FIG. 14, the second carrier member 20 is moved in the distal end direction (the direction of arrow X1) relative to the outer cylinder 22, so that the second support portion 50 on which the medical sheet 300 is placed is moved from the retracted position to an advanced position, and the second support portion 50 protrudes in the distal end direction (the direction of arrow X1) relative to the distal end of the first support portion 26. Specifically, in the moving process, the second shaft 48 is moved in the distal end direction relative to the first shaft 24. When the first shaft 24 reaches the distal end position of the relative movement range with respect to the outer cylinder 22, relative movement of the first shaft 24 in the distal end direction is restricted by the movement range regulation portion 94. On the other hand, even when the first shaft 24 reaches the distal end position of the relative movement range, the second carrier member 20 can further relatively move in the distal end direction with respect to the outer cylinder 22. Therefore, the user can move only the second carrier member 20 relative to the outer cylinder 22 in the distal end direction by further pushing the second carrier member 20 even after the first shaft 24 reaches the distal end position of the relative movement range with respect to the outer cylinder 22.

Accordingly, the second support portion 50 moves in the distal end direction (the direction of arrow X1) relative to the first support portion 26. At this time, when the pressing surface 58 of the carrier holding portion 54 (pressure-application portion 56) presses the outer edge surface of the medical sheet 300 in the distal end direction, the medical sheet 300 is entirely located further than the first support portion 26 in the distal end direction. In this moving process, the medical sheet 300 is moved to above the recipient site 402 of the heart 400 to bring the overhanging portion 302 of the medical sheet 300 into contact with the recipient site 402.

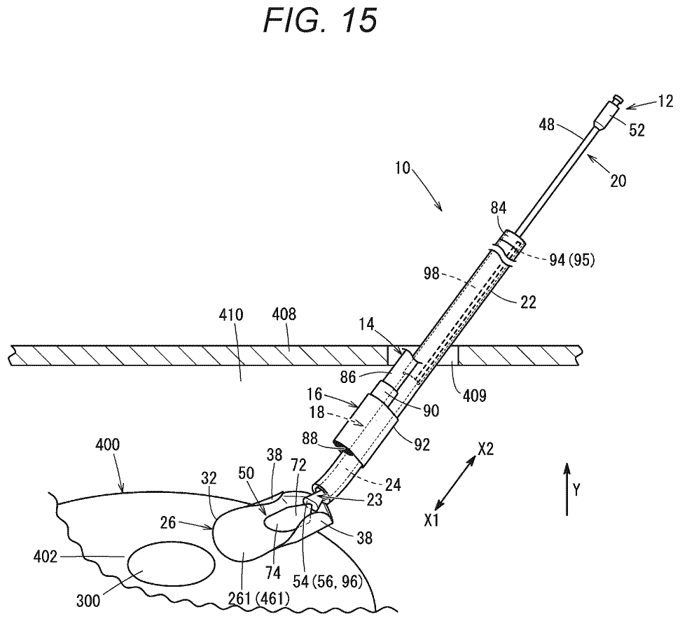

Subsequently, in the withdrawing process (S7 in FIG. 6), as illustrated in FIG. 15, the second carrier member 20 is moved from the second position to the first position to withdraw the second support portion 50 from between the recipient site 402 and the medical sheet 300. Accordingly, the medical sheet 300 entirely comes into contact with a front surface of the recipient site 402. This is the end of the transfer of the medical sheet 300 to the recipient site 402. Subsequently, the transfer instrument 10 is withdrawn from the chest 408 with the first support portion 26 and the second support portion 50 retracted into the outer cylinder 22. Note that, in the withdrawing process, the user can retract the first support portion 26 and the second support portion 50 in the outer cylinder 22 by operating the second carrier member 20. Specifically, the user holds and pulls the second shaft 48 in the proximal end direction. Accordingly, the third engagement portion 96 can be engaged with the first shaft 24. By further pulling the second shaft 48 in the proximal end direction in a state where the third engagement portion 96 is engaged with the first shaft 24, the first shaft 24 can be moved in the proximal end direction as the second shaft 48 moves in the proximal end direction. In this manner, the first carrier member 18 and the second carrier member 20 can be retracted in the outer cylinder 22.

The present embodiment has the following effects.

Since the transfer instrument 10 includes the second carrier member 20 movable in the axial direction relative to the first carrier member 18, it is possible to transfer the medical sheet 300 from above the first support portion 26 to the recipient site 402 of the living body using the second carrier member 20 without using another device (forceps or the like). It is therefore possible to efficiently transfer the medical sheet 300 to the recipient site 402.

As illustrated in FIG. 2, the first support portion 26 is formed of a flexible sheet including the front surface 461 and the back surface 462. The first support portion 26 includes the pair of first protrusions 38 protruding upward from both sides of the first support surface 261 in the width direction orthogonal to the direction in which the first shaft 24 moves. With this configuration, as illustrated in FIG. 14, when the first support portion 26 is retracted into the outer cylinder 22, the back surfaces 462 of the pair of first protrusions 38 curved and deformed into a convex shape come into contact with each other. The pair of first protrusions 38 are displaced toward the first support surface 261, and the first support portion 26 is curved and deformed into a heart shape. Accordingly, the first support portion 26 can be retracted with the heart shape in the outer cylinder 22, so that it is possible to effectively reduce the possibility of breakage of the medical sheet 300 held by the first support portion 26 as compared with a case where the first support portion 26 is deformed into a shape other than the heart shape. Since the first support portion 26 can be smoothly and compactly retracted into the outer cylinder 22, the diameter of the outer cylinder 22 can be reduced as compared with a configuration where the first support portion 26 is not curved and deformed into a heart shape.

According to the present embodiment, when the second carrier member 20 moves between the first position and the second position, the second shaft 48 protrudes in the proximal end direction from the proximal-end opening 81 of the outer cylinder 22. Therefore, the preparing process, the unfolding process, the moving process, and the like can be achieved by the user pushing, in the distal end direction, the portion of the second shaft 48 protruding from the proximal-end opening 81 of the outer cylinder 22. Note that, in the moving process, the movement range regulation portion 94 restricts the relative movement of the first shaft 24 in the distal end direction with respect to the outer cylinder 22.

Further, in the retracting process, the withdrawing process, and the like, the carrier holding portion 54, which is the third engagement portion 96, can be engaged with the distal-end opening 25 of the first shaft 24 by relatively moving the second shaft 48 in the proximal end direction. The first carrier member 18 and the second carrier member 20 can be integrally moved in the proximal end direction by further relatively moving the second shaft 48 in the proximal end direction in a state where the carrier holding portion 54 is engaged with the distal-end opening 25.

Note that, unlike the present embodiment, in a case where not only the second shaft 48 but also the first shaft 24 protrudes from the proximal-end opening 81 of the outer cylinder 22 at a start stage of the retracting process, the withdrawing process, and the like, there is a possibility that the user may be confused as to which one of the first shaft 24 and the second shaft 48 should be operated. In addition, unlike the present embodiment, in a case where the first shaft 24 protrudes from the proximal-end opening 81 of the outer cylinder 22 and there is no third engagement portion 96, the first shaft 24 is not engaged with the second carrier member 20. In a case where the first shaft 24 is not engaged with the second carrier member 20, even if only the second shaft 48 is pulled, the first support portion 26 cannot be retracted into the outer cylinder 22. As a result, the user cannot efficiently perform the retracting process, the withdrawing process, and the like.

In this regard, according to the present embodiment, the first shaft 24 does not protrude in the proximal end direction from the proximal-end opening 81 of the outer cylinder 22. On the other hand, the second shaft 48 protrudes in the proximal end direction from the proximal-end opening 81 of the outer cylinder 22. Therefore, the user can operate the second shaft 48 without being confused in the retracting process, the withdrawing process, and the like. As described above, the transfer instrument 10 of the present embodiment makes it relatively easy for the user to understand a target portion to be operated, and can help prevent an erroneous operation by the user. It is therefore possible to efficiently transfer the medical sheet 300 to the treatment site.

As illustrated in FIG. 10, in a state where the first support portion 26 is retracted in the outer cylinder 22, relative movement between the first shaft 24 and the second shaft 48 in the axial direction is prevented by the movement regulation portion 23 bringing the distal portion of the second shaft 48 and the first support portion 26 into contact with each other. As a result, when the user moves the second carrier member 20 in the distal end direction, the first carrier member 18 also moves in the distal end direction together with the second carrier member 20. As a result, the second support portion 50 and the medical sheet 300 are prevented from protruding from the distal-end opening 80 of the outer cylinder 22. As a result, the medical sheet 300 retracted in the outer cylinder 22 is prevented from being pushed and broken by the distal portion of the second shaft 48. Therefore, the medical sheet 300 can be efficiently transferred to the recipient site 402 without breakage.

The movement regulation portion 23 is the pressure-application portion 56 constituting the distal portion of the second shaft 48. In a state where the first support portion 26 is retracted in the outer cylinder 22, the pressure-application portion 56 presses the first support portion 26 against the lumen 78 (inner surface) of the outer cylinder 22. As a result, the pressure-application portion 56 constituting the movement regulation portion 23 and the first support portion 26 are brought into contact or relatively close contact with each other in the radial direction, so that relative displacement between the first support portion 26 and the second shaft 48 is restricted. The second shaft 48 can be made movable in the axial direction with respect to the first shaft 24 in a state where the first support portion 26 protrudes from the distal portion of the outer cylinder 22.

Since the pressure-application portion 56 includes an elastic body, the pressure-application portion 56 can be effectively brought into contact or relatively close contact with the first support portion 26. Even if the second shaft 48 is pushed in the distal end direction (the direction of arrow X1) when the pressure-application portion 56 is retracted into the outer cylinder 22, the second shaft 48 is bent and a pressing force is less likely to be transmitted to the pressure-application portion 56 since the pressure-application portion 56 is an elastic body. Therefore, the user can easily notice an erroneous operation.

In a cross section orthogonal to the axis of the second shaft 48, the pressure-application portion 56 includes the pair of linear portions 621, 622 and the pair of convex-shaped portions 641, 642 provided on both sides of the pair of linear portions 621, 622. The first length D1 along the extending direction of the linear portions 621, 622 is longer than the second length D2 (see FIG. 5). As a result, when the first support portion 26 is retracted into the outer cylinder 22, when the first support portion 26 is pushed toward the outer cylinder 22 with the pair of convex-shaped portions 641, 642 provided on both sides of the linear portions 621, 622, the first support portion 26 and the carrier holding portion 54 can be brought into closer contact with each other.

As illustrated in FIG. 1, the second support portion 50 having a sheet shape and including the second support surface 74 capable of holding the medical sheet 300 is provided to the distal portion of the second shaft 48. The second support portion 50 is movable relative to the first support portion 26 between the retracted position where the second support portion 50 is placed on the first support surface 261 and the advanced position located adjacent to the distal end (in the direction of arrow X1) relative to the first support surface 261. With this configuration, the medical sheet 300 can be moved along the first support surface 261 of the first support portion 26 by the second support portion 50 including the second support surface 74.

As illustrated in FIG. 9, at the retracted position (first position), the second support portion 50 is retracted in the outer cylinder 22 together with the first support portion 26 with the second support portion 50 curved and deformed. With this configuration, even the second support portion 50 having a width dimension can be suitably retracted inside the outer cylinder 22 together with the first support portion 26 in the retracting process.

The carrier holding portion 54 that holds a proximal portion of the second support portion 50 is provided to the distal portion of the second shaft 48. Unlike the present embodiment, in a case where the carrier holding portion 54 does not have a vertically symmetrical shape and the second support portion 50 is held at a lower end of the carrier holding portion 54, the user rotates the second shaft 48 to reverse a vertical direction of the second support portion 50, and the second support portion 50 is disposed above the carrier holding portion 54. In this case, the second support portion 50 rises with respect to the first support surface 261 of the first support portion 26, and the first support portion 26 and the second support portion 50 are separated in the vertical direction.

On the other hand, in the present embodiment, as illustrated in FIG. 5, the carrier holding portion 54 is formed in line symmetry (vertical symmetry) with respect to the center line C of the carrier holding portion 54, in the cross section orthogonal to the axis of the second shaft 48. Further, the carrier holding portion 54 holds the second support portion 50 on the center line C. Therefore, even when the vertical direction of the second support portion 50 is reversed by the user rotating the second shaft 48, rising of the second support portion 50 from the first support surface 261 of the first support portion 26 can be suppressed as compared with the configuration in which the second support portion 50 is held at the lower end of the carrier holding portion 54. As a result, the medical sheet 300 can be stably subjected to a transplant procedure by the second support portion 50.

In a cross section orthogonal to the protruding direction of the first protrusion 38 and orthogonal to the first support surface 261, each cross section of the first protrusions 38 has a shape in which the intermediate portion 443 between the free end 442 and the fixed end 441 of the first protrusion 38 bulges in a convex shape in a direction away from the first support surface 261. With this configuration, when the first support portion 26 is retracted into the outer cylinder 22, the first support portion 26 can be reliably curved and deformed into a heart shape. As a result, it is possible to prevent breakage of the medical sheet 300 due to the deformation of the first support portion 26 into a shape other than the heart shape.

Unlike the present embodiment, in a case where the intermediate portion 443 has a curved shape so as to be concaved toward the first support surface 261 on the back surface 462 of the first support portion 26, the first support portion 26 may not be curved and deformed into an appropriate heart shape in the outer cylinder 22 in some rare cases. For example, since the free end 442 (front surface 461) of one first protrusion 38 rides on the fixed end 441 (back surface 462) of another first protrusion 38 that is curved and deformed, the free end 442 is bent in a V shape toward the inner surface of the outer cylinder 22 in some rare cases. In the present embodiment, since the intermediate portion 443 has a shape bulging in a convex shape in a direction away from the first support surface 261, it is possible to prevent the V-shaped bending of the first support portion 26.

By forming the intermediate portion 443 of the pair of first protrusions 38 in an arc shape, the first protrusion 38 having the shape bulging outward can be easily formed.

Since the curvature R of the intermediate portion 443 of the first protrusion 38 increases in the proximal end direction (the direction of arrow X2) of the first support portion 26, the interval W in the width direction between the first protrusions 38 on the distal end side of the first protrusion 38 can be widened. As a result, the medical sheet 300 is easily placed on the first support surface 261 of the first support portion 26 through the distal-end support portion 42.

As illustrated in FIG. 3, the interval W between the pair of bent portions 444 in the width direction of the first support portion 26 decreases in the proximal end direction. Therefore, when the first support portion 26 is retracted into the outer cylinder 22 from the proximal end side, the pair of first protrusions 38 can be suitably curved and deformed in a conical shape by being in contact with the outer cylinder 22. Accordingly, interference between the proximal ends of the pair of bent portions 444 is suppressed, and thus the first support portion 26 can be smoothly retracted into the outer cylinder 22 from the proximal end side.

The distal portion of the first support portion 26 has an arc shape connecting the pair of second protrusions 40 when viewed from a direction orthogonal to the first support surface 261. Therefore, sliding resistance between the first support portion 26 and the outer cylinder 22 can be reduced when the first support portion 26 is retracted into the outer cylinder 22, as compared with a configuration in which the distal portion of the first support portion 26 has a shape having a corner portion. When the medical sheet 300 is placed on the first support portion 26 by moving the first support portion 26 in the distal end direction (the direction of arrow X1), the work can be smoothly performed by gradually inserting the first support portion 26 into the interface between the medical sheet 300 and the Petri dish 401 (container).

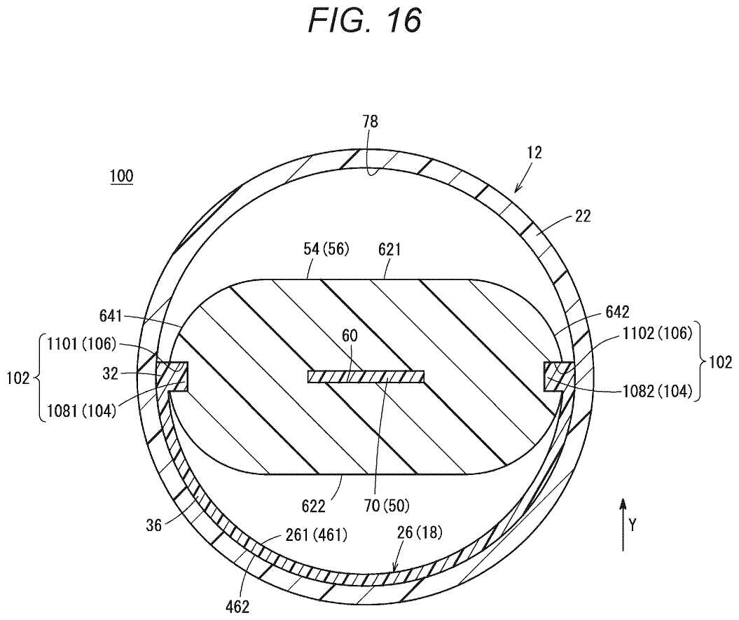

As illustrated in FIG. 16, a transfer instrument 100 according to a first modification includes a movement regulation portion 102. Note that, in the present modification, the same components as those of the above-described transfer instrument 10 are denoted by the same reference numerals, and the detailed description of the same components will be omitted. The same applies to transfer instruments 120, 130, 150, 160 according to second to fifth modifications described later.

The movement regulation portion 102 includes a first fitting portion 104 provided to the first support portion 26 and a second fitting portion 106 provided to the carrier holding portion 54 at the distal portion of the second shaft 48.

The first fitting portion 104 includes a pair of first projections 1081, 1082 protruding upward from the first support surface 261 of the first support portion 26. The first projections 1081 and 1082 each are disposed at equal distances from a center of the first support portion 26 in a width direction. When viewed from a direction orthogonal to the width direction of the first support portion 26, for example, cross sections of the first projections 1081, 1082 are rectangular, for example. The cross sections of the first projections 1081, 1082 may have other shapes (semicircular shapes or the like).

The second fitting portion 106 is disposed at each of both end portions in a width direction (convex-shaped portions 641, 642) of the carrier holding portion 54 (pressure-application portion 56). The second fitting portion 106 includes recesses 1101, 1102 recessed from outer surfaces of the convex-shaped portions 641, 642. The recesses 1101, 1102 are recessed in a width direction from the outer surfaces of the convex-shaped portions 641, 642. When viewed from an axial direction of the carrier holding portion 54, cross sections of the recesses 1101, 1102 have rectangular shapes corresponding to the first projections 1081, 1082. The cross sections of the recesses 1101, 1102 may have other shapes (semicircular or the like).

In a state where the first support portion 26 is retracted into the outer cylinder 22 and the first support portion 26 is curved and deformed, the first projections 1081, 1082 of the first fitting portion 104 constituting the movement regulation portion 102 are respectively fitted into the recesses 1101, 1102 of the second fitting portion 106. As a result, relative displacement between the first support portion 26 and the carrier holding portion 54 is restricted, and accordingly, relative movement of the second shaft 48 in the axial direction with respect to the first shaft 24 is restricted.

As described above, in the transfer instrument 100 according to the first modification, when the first fitting portion 104 and the second fitting portion 106 in the movement regulation portion 102 are fitted in a state (first position) where the first support portion 26 is retracted in the outer cylinder 22, relative movement in the axial direction between the first shaft 24 including the first support portion 26 and the second shaft 48 is prevented. When fitting between the first fitting portion 104 and the second fitting portion 106 of the movement regulation portion 102 is released in a state where the first support portion 26 protrudes from the distal portion of the outer cylinder 22 (second position), the second shaft 48 becomes movable in the distal end direction (the direction of arrow X1) with respect to the first support portion 26.

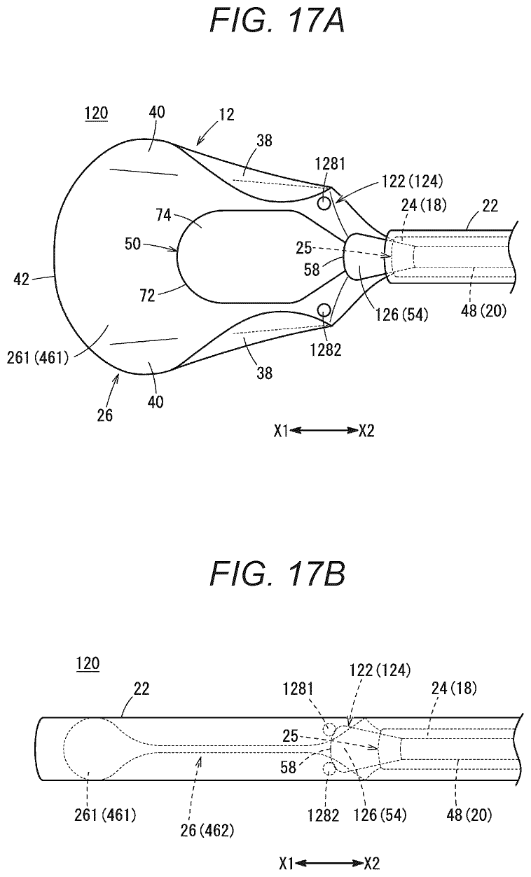

As illustrated in FIG. 17A, the transfer instrument 120 according to the second modification includes a movement regulation portion 122. The movement regulation portion 122 includes a first engagement portion 124 and a second engagement portion 126. The first engagement portion 124 is provided on the front surface 461 of the first support portion 26 including the first support surface 261. The second engagement portion 126 is configured by the carrier holding portion 54 at the distal portion of the second shaft 48.

The first engagement portion 124 includes a pair of second projections 1281, 1282 projecting upward from the first support surface 261. The second projections 1281, 1282 each are disposed at equal distances from a center of the first support portion 26 in the width direction. The second engagement portions 126 are disposed on both sides of the distal end of the carrier holding portion 54.

As illustrated in FIG. 17B, in a state where the first support portion 26 is retracted into the outer cylinder 22 and the first support portion 26 is curved and deformed, the second projections 1281, 1282 of the first engagement portion 124 and the second engagement portion 126 face each other in the axial direction of the outer cylinder 22, and the first engagement portion 124 and the second engagement portion 126 are engaged with each other in the axial direction.

As described above, in the transfer instrument 120 according to the second modification, when the first engagement portion 124 constituting the movement regulation portion 122 and the second engagement portion 126 are engaged in the axial direction in a state (first position) where the first support portion 26 is retracted in the outer cylinder 22 illustrated in FIG. 17B, relative movement in the axial direction between the first shaft 24 including the first support portion 26 and the second shaft 48 is prevented. In a state (second position) where the first support portion 26 illustrated in FIG. 17A protrudes from the distal-end opening 80 of the outer cylinder 22, the first engagement portion 124 and the second engagement portion 126 do not face each other in the axial direction as the first support portion 26 is unfolded, and the engagement between the first engagement portion 124 and the second engagement portion 126 is released. As a result, the second shaft 48 becomes movable in the distal end direction (the direction of arrow X1) with respect to the first support portion 26.

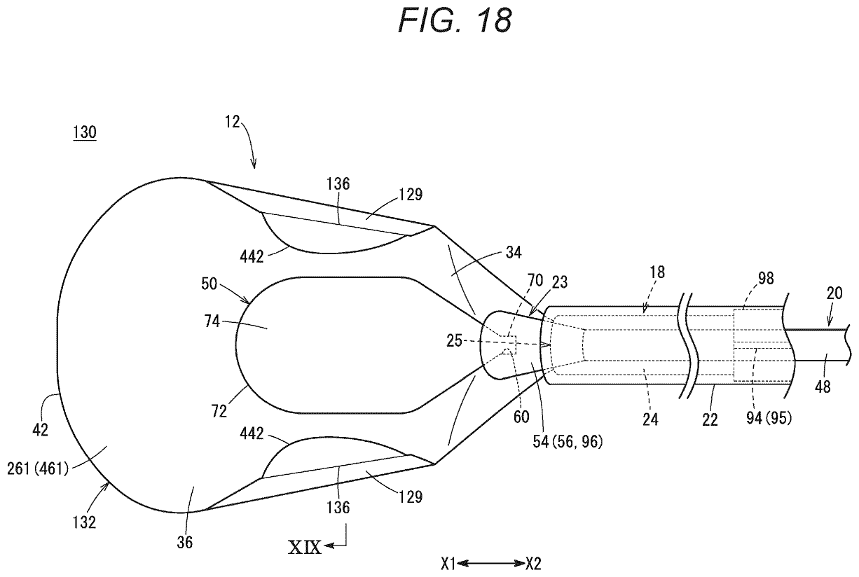

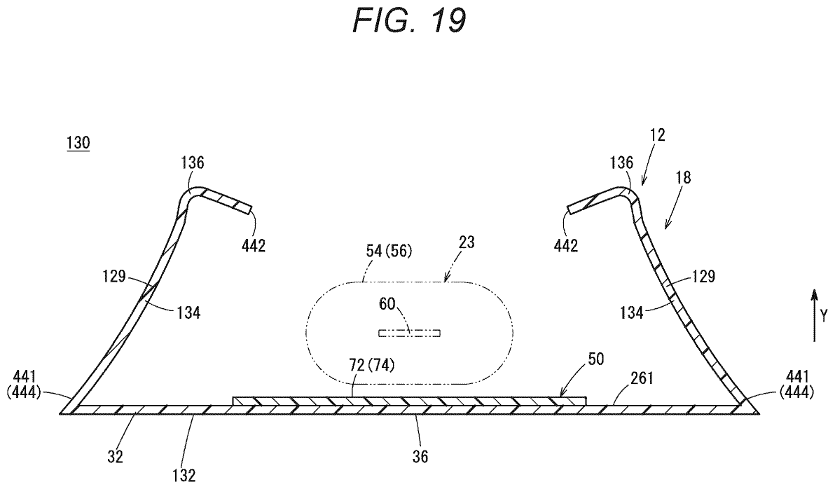

As illustrated in FIGS. 18 and 19, the transfer instrument 130 according to the third modification includes a first support portion 132. Protrusions 129 provided on both sides in a width direction of the first support portion 132 each have an intermediate portion 134 between the free end 442 and the fixed end 441. The intermediate portion 134 has a shape bulging in a convex shape in a direction away from the first support surface 261. The intermediate portion 134 includes an intermediate bent portion 136 bent so as to form a mountain fold on the back surface 462. The intermediate bent portion 136 is disposed closer to the free end 442 in the intermediate portion 134.

As described above, in the transfer instrument 130 according to the third modification, by providing the intermediate bent portion 136 in the protrusion 129 of the first support portion 132, it is possible to easily form the protrusion 129 having the shape bulging in the convex shape in the direction away from the first support surface 261.

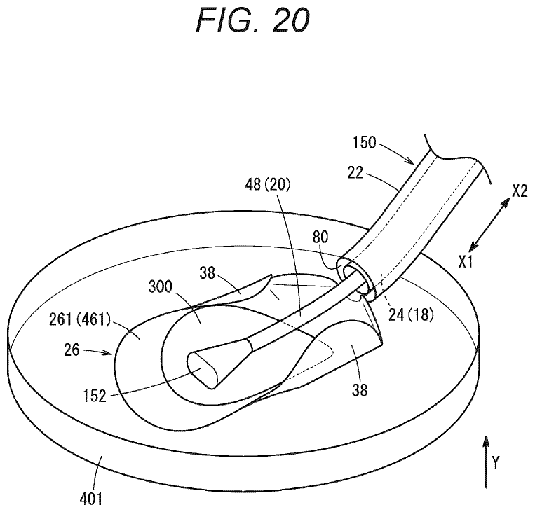

Although the case where the second support portion 50 is attached to the distal portion of the second shaft 48 has been described, the present disclosure is not limited to the second support portion 50 being attached to the distal portion of the second shaft 48. For example, as in the transfer instrument 150 according to the fourth modification illustrated in FIG. 20, a pressing body 152 may be provided at a distal portion of the second shaft 48. The pressing body 152 has, for example, a flattened cross-sectional shape including a flat bottom surface. The medical sheet 300 placed on the first support surface 261 of the first support portion 26 can be pressed downward by the bottom surface of the pressing body 152 to push out the medical sheet 300 in the distal end direction (the direction of arrow X1) along the first support surface 261 of the first support portion 26.

By increasing a width of at least a part of the pressing body 152 to be larger than the inner diameter D24 (see FIG. 3) of the first shaft 24, the pressing body 152 can be made function as the third engagement portion 96.

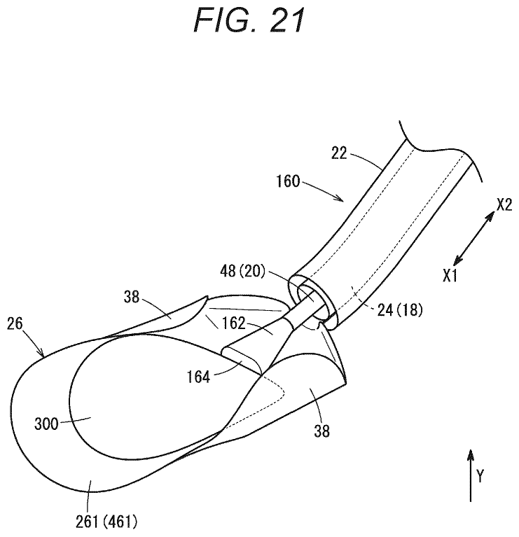

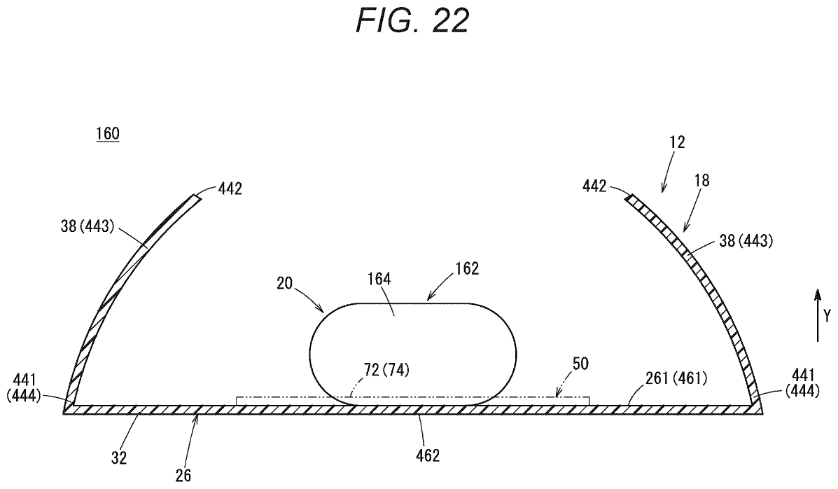

As in the transfer instrument 160 according to the fifth modification illustrated in FIGS. 21 and 22, a pressing body 162 may be provided to the distal portion of the second shaft 48. A distal end of the pressing body 162 includes a pressing surface 164. The pressing surface 164 is a flat surface orthogonal to an axis of the pressing body 162. A proximal end of the pressing body 162 is connected to the distal portion of the second shaft 48. The medical sheet 300 placed on the first support surface 261 of the first support portion 26 can be pressed by the pressing surface 164 of the pressing body 162 to push out the medical sheet 300 in the distal end direction (the direction of arrow X1) along the first support surface 261 of the first support portion 26.

Further, by increasing a width of at least a part of the pressing body 162 to be larger than the inner diameter D24 (see FIG. 3) of the first shaft 24, the pressing body 162 can be made function as the third engagement portion 96.

### Second Embodiment

Hereinafter, a second embodiment will be described. Note that elements already described in the first embodiment are denoted by the same reference numerals as those in the first embodiment also in the present embodiment unless otherwise specified. Further, in the second embodiment, the description overlapping with the first embodiment will be appropriately omitted.

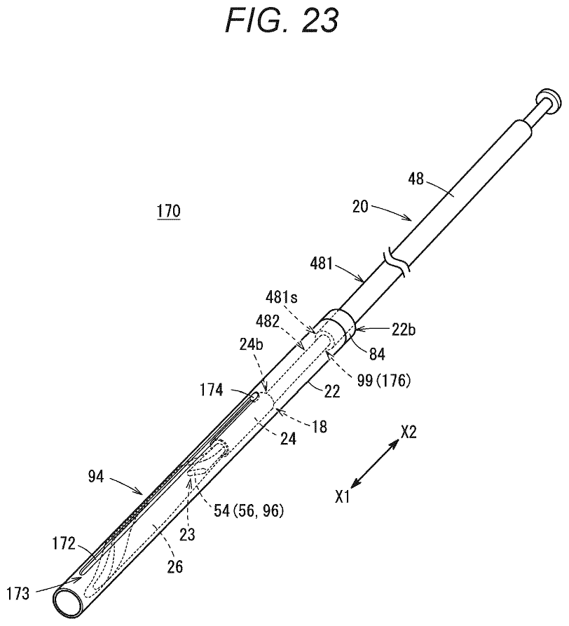

Each of FIGS. 23 to 26 is a perspective view of a transfer instrument 170 according to the second embodiment.

The transfer instrument 170 includes an outer cylinder 22, a first carrier member 18, a second carrier member 20, a movement regulation portion 23, and a movement range regulation portion 94. The first carrier member 18 includes a first shaft 24. The second carrier member 20 includes a carrier holding portion 54 (third engagement portion 96) and a second shaft 48.

The movement range regulation portion 94 of the present embodiment includes a notch 172 and a projection 174. The notch 172 is formed in one of the outer cylinder 22 or the first shaft 24 along an axial direction (X1, X2). The projection 174 is formed on another one of the outer cylinder 22 or the first shaft 24. Note that, FIGS. 23 to 26 illustrate an example of a case in which the notch 172 is formed in the outer cylinder 22 and the projection 174 is formed in the first shaft 24.

The projection 174 is fitted in the notch 172, and slides along the notch 172 in an axial direction of the outer cylinder 22. In this case, a relative movement range between the outer cylinder 22 and the first shaft 24 along the axial direction of the outer cylinder 22 is limited on the basis of a length of the notch 172. A distal end position of the relative movement range of the first shaft 24 is defined by a position of a distal end 173 on the distal end direction (X1) side of the notch 172.

FIG. 23 illustrates the transfer instrument 170 with the second carrier member 20 being at a first position. By pushing the second shaft 48 in a distal end direction from the state of FIG. 23, the first carrier member 18 and the second carrier member 20 can be relatively moved in the distal end direction integrally with respect to the outer cylinder 22.

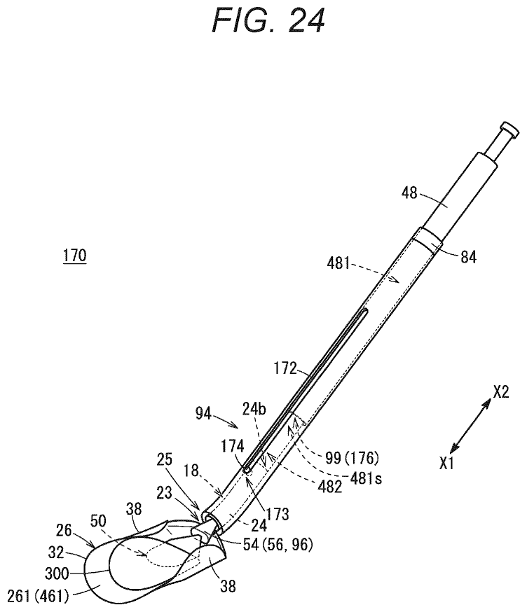

FIG. 24 illustrates the transfer instrument 170 with the projection 174 reaching the distal end 173 of the notch 172. In the state of FIG. 24, the second carrier member 20 has reached a second position, and each of the first support portion 26 and the second support portion 50 is unfolded. When the second shaft 48 is further pushed in the distal end direction in the state of FIG. 24, the first carrier member 18 does not move relative to the outer cylinder 22. On the other hand, when the second shaft 48 is further pushed in the distal end direction in the state of FIG. 24, the second carrier member 20 moves relative to the outer cylinder 22 in the distal end direction.

The transfer instrument 170 further includes a fourth engagement portion 99. The fourth engagement portion 99 defines the distal end position of the relative movement range of the second carrier member 20 that relatively moves in the distal end direction (X1) with respect to the outer cylinder 22. More specifically, the fourth engagement portion 99 of the present embodiment includes a stepped portion 176 described below.

The second shaft 48 of the present embodiment includes a first portion 481 and a second portion 482. In the second shaft 48, the first portion 481 is a portion extending in a distal end direction from a proximal portion of the second shaft 48 by a predetermined length. In the second shaft 48, the second portion 482 is a portion extending in the distal end direction from a distal end of the first portion 481.

A diameter of the first portion 481 is larger than a diameter of the second portion 482. As a result, the stepped portion 176 is formed at a boundary portion between the first portion 481 and the second portion 482 in the second shaft 48. The stepped portion 176 includes an end surface 481s on the distal end direction side of the first portion 481.

Note that, a diameter of the first portion 481 is smaller than an inner diameter of the outer cylinder 22. Therefore, insertion of the first portion 481 into the outer cylinder 22 is not inhibited. Further, a diameter of the second portion 482 is smaller than the inner diameter D24 (see FIG. 3) of the first shaft 24. Therefore, insertion of the second portion 482 into the first shaft 24 is not inhibited.

As described above, in a state where the projection 174 reaches the distal end 173 of the notch 172, the first carrier member 18 cannot move relative to the outer cylinder 22 in the distal end direction. On the other hand, even in a state where the projection 174 reaches the distal end 173 of the notch 172, the second carrier member 20 is relatively movable in the distal end direction with respect to the outer cylinder 22.

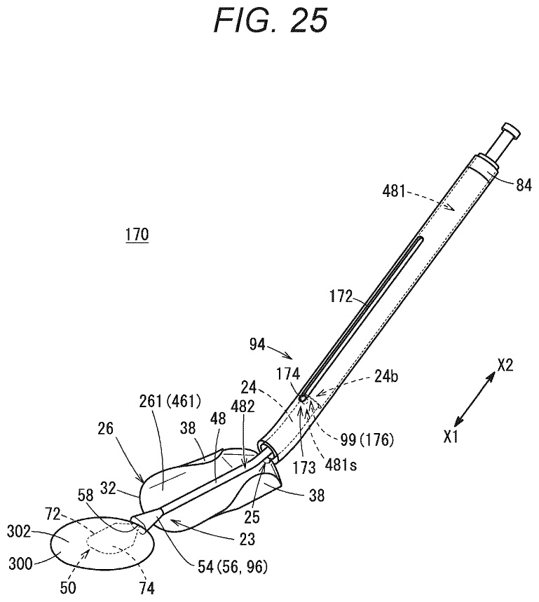

Therefore, by further pushing the second shaft 48 in the distal end direction after the projection 174 reaches the distal end 173 of the notch 172, the stepped portion 176 and a proximal portion 24b of the first shaft 24 approach each other. Accordingly, the stepped portion 176 and the proximal portion 24b of the first shaft 24 can be brought into contact with each other. More specifically, the end surface 481s and the proximal portion 24b can be brought into contact with each other (see also FIG. 25).

The proximal portion 24b of the first shaft 24 in contact with the stepped portion 176 prevents the second shaft 48 from relatively moving in the distal end direction with respect to the outer cylinder 22. That is, a position of the second carrier member 20 in a state where the stepped portion 176 and the proximal portion 24b of the first shaft 24 are in contact with each other is defined as the distal end position of the relative movement range of the second carrier member 20.

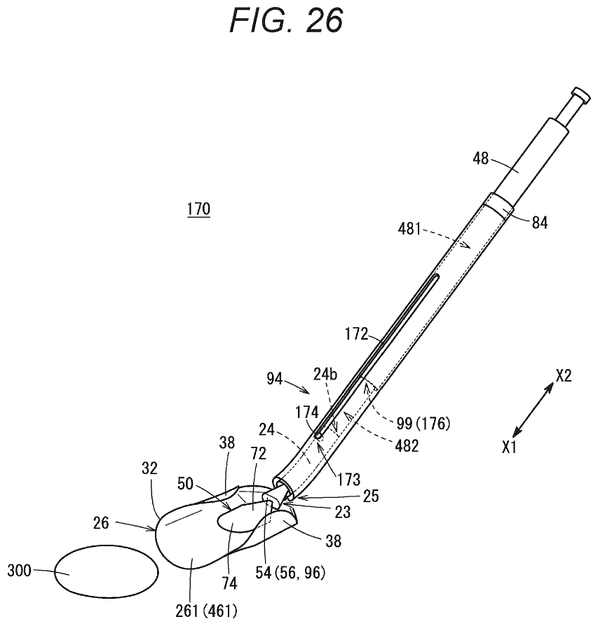

A width W54 of at least a part of the carrier holding portion 54 which is the third engagement portion 96 is larger than the inner diameter D24 of the first shaft 24 (see also FIG. 3). Therefore, by pulling back the second shaft 48 in the proximal end direction (X2) from the state of FIG. 25, the first shaft 24 can be engaged with the carrier holding portion 54 which is the third engagement portion 96 (see also FIG. 26). By further pulling back the second shaft 48 in the proximal end direction in a state where the first shaft 24 and the carrier holding portion 54 are engaged with each other, the transfer instrument 170 can return to the state illustrated in FIG. 23.

The present embodiment has the following effects.

Similarly to the first embodiment, when the second carrier member 20 is moved from the first position to the second position, the first carrier member 18 and the second carrier member 20 are integrally moved in the distal end direction by relatively moving the second shaft 48 in the distal end direction with respect to the outer cylinder 22. Therefore, the preparing process, the unfolding process, the moving process, and the like (see FIG. 6) can be achieved by the user pushing, in the distal end direction, the portion of the second shaft 48 protruding from a proximal-end opening 81 of the outer cylinder 22. Note that, in the moving process, the movement range regulation portion 94 restricts the relative movement of the first shaft 24 in the distal end direction with respect to the outer cylinder 22.

Further, when the second carrier member 20 is moved from the second position to the first position in the retracting process, the withdrawing process, and the like (see FIG. 6), the second shaft 48 is relatively moved in the proximal end direction. As a result, the carrier holding portion 54 which is the third engagement portion 96 can be engaged with the distal-end opening 25 of the first shaft 24. The first carrier member 18 and the second carrier member 20 can be integrally moved in the proximal end direction by further relatively moving the second shaft 48 in the proximal end direction in a state where the carrier holding portion 54 is engaged with the distal-end opening 25.

Moreover, according to the present embodiment, similarly to the first embodiment, the first shaft 24 does not protrude in the proximal end direction from the proximal-end opening 81 of the outer cylinder 22. Therefore, the user can operate the second shaft 48 without being confused in the retracting process, the withdrawing process, and the like. As described above, the transfer instrument 170 of the present embodiment makes it relatively easy for the user to understand a target portion to be operated, and can help prevent an erroneous operation by the user. It is therefore possible to efficiently transfer the medical sheet 300 to the treatment site.

The movement range regulation portion 94 includes the notch 172 and the projection 174. The notch 172 is formed in one of the outer cylinder 22 or the first shaft 24 along the axial direction of the outer cylinder 22. The projection 174 is formed on another one of the outer cylinder 22 or the first shaft 24. The projection 174 can be integrally molded with the outer cylinder 22 or the first shaft 24.

For example, the fourth engagement portion 99 according to the second embodiment is not limited to the stepped portion 176. For example, the fourth engagement portion 99 may be a protrusion protruding from the second shaft 48 along a direction perpendicular to the axial direction of the outer cylinder 22. In this case, the protrusion comes into contact with the proximal portion 24b of the first shaft 24. Even in such a case, the distal end position of the relative movement range of the second carrier member 20 can be defined by the fourth engagement portion 99. Further, in this case, it is not necessary to make the diameter different between a portion of the second shaft 48 on the distal end direction side relative to the fourth engagement portion 99 and a portion of the second shaft 48 on the proximal end direction side relative to the fourth engagement portion 99.

In addition, the fourth engagement portion 99 may be located on the proximal end direction side relative to a proximal portion 22b of the outer cylinder 22. For example, in a state where the second shaft 48 is located on the proximal-most end side in the movable range with respect to the outer cylinder 22, the stepped portion 176 may be located on the proximal end direction side relative to the proximal portion 22b of the outer cylinder 22. In this case, an outer diameter of the first portion 481 of the second shaft 48 is larger than an inner diameter of the outer cylinder 22. As a result, when the second carrier member 20 is relatively moved in the distal end direction with respect to the outer cylinder 22, the stepped portion 176 and the proximal portion 22b of the outer cylinder 22 can be brought into contact with each other. Even in such a case, the distal end position of the relative movement range of the second carrier member 20 can be defined by the fourth engagement portion 99.

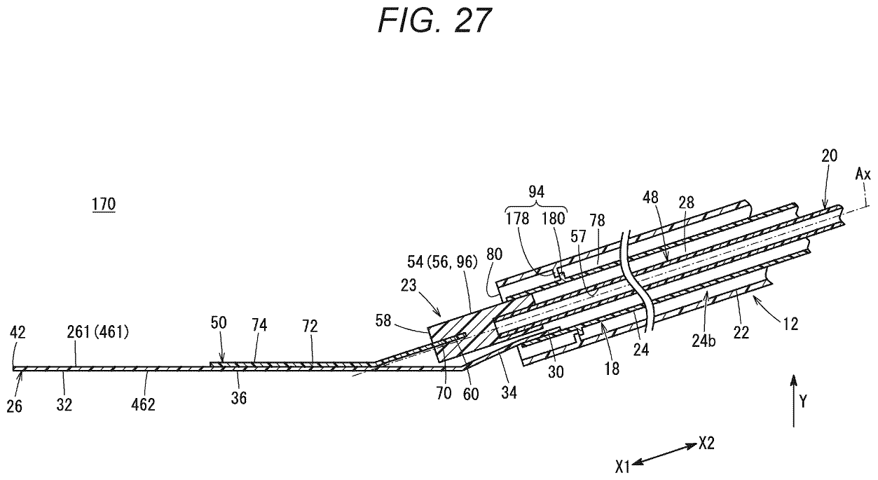

Further, as illustrated in FIG. 27, the movement range regulation portion 94 may include an outer projection 178 and an inner projection 180. The outer projection 178 is formed in the outer cylinder 22. The outer projection 178 protrudes inward of the outer cylinder 22 so as to partially narrow the inner diameter of the outer cylinder 22. On the other hand, the inner projection 180 is formed in the first shaft 24. The inner projection 180 protrudes outward of the first shaft 24 so as to partially expand the outer diameter of the first shaft 24. The inner projection 180 is located on the proximal end direction side relative to the outer projection 178.

The outer projection 178 interferes with (is engaged with) the inner projection 180 when the first shaft 24 moves relative to the outer cylinder 22 in the distal end direction. The inner projection 180 interferes with the outer projection 178 to prevent the first shaft 24 from moving relative to the outer cylinder 22 in the distal end direction. In this way, a position of the second carrier member 20 in the state where the outer projection 178 and the inner projection 180 interfere with each other can be defined as the distal end position of the relative movement range of the second carrier member 20.

Furthermore, the first embodiment and the modifications according to the first embodiment may be combined with the second embodiment within a range not contradictory.

The detailed description above describes embodiments of a transfer instrument for transferring a medical sheet to a treatment site of a living body. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A transfer instrument for transferring a medical sheet to a treatment site of a living body, the transfer instrument comprising:
an outer cylinder;
a first carrier member including a first shaft disposed inside the outer cylinder to be movable along an axial direction of the outer cylinder, and a first support portion having a sheet shape, disposed at a distal portion of the first shaft, and including a support surface configured to hold the medical sheet when the distal portion of the first shaft is disposed in a distal end direction with respect to a distal portion of the outer cylinder; and
a second carrier member including a second shaft extending along the first shaft and provided movably along the first shaft, wherein
the first support portion is retracted into the outer cylinder in a state of being curved and deformed at a first position where the first and second shafts are moved in a proximal end direction with respect to the outer cylinder such that the first support portion is retracted into the outer cylinder,
the first support portion is unfolded by being exposed from the outer cylinder in the distal end direction at a second position where the first and second shafts are moved in the distal end direction with respect to the outer cylinder such that the first support portion protrudes from a distal-end opening of the outer cylinder, the first shaft is shorter than the outer cylinder in the axial direction, and the first shaft is entirely located on the distal end direction side relative to a proximal-end opening of the outer cylinder at the first position,
the second shaft is longer than the outer cylinder in the axial direction and protrudes from the proximal-end opening of the outer cylinder, and
a movement regulation portion that regulates relative movement of the first shaft and the second shaft in the axial direction between from the first position to the second position;
a movement range regulation portion that defines a distal end position of a relative movement range of the first shaft in the axial direction with respect to the outer cylinder; and
an engagement portion that is provided to the second carrier member and is engaged with the first carrier member to move the first carrier member in the proximal end direction when the second carrier member moves from the second position toward the first position.

2. The transfer instrument according to claim 1, wherein
the movement range regulation portion includes a coupling member having a string shape or wire shape and connected to the outer cylinder and the first carrier member.

3. The transfer instrument according to claim 2, wherein
one end of the coupling member is connected to a proximal portion of the outer cylinder, and another end of the coupling member is connected to a proximal portion of the first shaft.

4. The transfer instrument according to claim 2, wherein
the coupling member is a resin film.

5. The transfer instrument according to claim 1, wherein
the movement range regulation portion includes a notch formed in one of the outer cylinder or the first shaft along the axial direction, and a projection that is formed on another one of the outer cylinder or the first shaft and slides along the notch.

6. The transfer instrument according to claim 1, wherein
the movement regulation portion comes into contact with the first support portion and a distal portion of the second shaft between from the first position to the second position, to regulate relative movement in the axial direction between the first shaft and the second shaft.

7. The transfer instrument according to claim 6, wherein
the movement regulation portion is a pressure-application portion configured by the distal portion of the second shaft; and
relative displacement between the first support portion and the second shaft is restricted by the pressure-application portion pressing the first support portion against an inner surface of the outer cylinder in a state where the first support portion is retracted in the outer cylinder.

8. The transfer instrument according to claim 6, wherein
the movement regulation portion includes a first fitting portion provided to the first support portion, and a second fitting portion provided to the distal portion of the second shaft;
relative displacement between the first support portion and the second shaft is restricted by fitting between the first fitting portion and the second fitting portion in a state where the first support portion is retracted in the outer cylinder; and
fitting between the first fitting portion and the second fitting portion is released by the first support portion being unfolded at the second position.

9. The transfer instrument according to claim 6, wherein
the movement regulation portion includes a first engagement portion provided on a front surface of the first support portion including the support surface, and a second engagement portion formed at the distal portion of the second shaft;
in a state where the first support portion is retracted in the outer cylinder, relative displacement between the first support portion and the second shaft is restricted by the first engagement portion and the second engagement portion facing each other in the axial direction of the second shaft and the first engagement portion and the second engagement portion being engaged with each other in the axial direction; and
engagement between the first engagement portion and the second engagement portion is released without the first engagement portion and the second engagement portion facing each other in the axial direction, by the first support portion being unfolded at the second position.

10. The transfer instrument according to claim 1, wherein
the first shaft has a tubular shape;
the second shaft includes a shaft body inserted through the first shaft;
the engagement portion extends from a distal portion of the shaft body; and
at least a part of the engagement portion is located on the distal end direction side relative to the first shaft, and has a larger dimension in a direction perpendicular to the axial direction than an inner diameter of the first shaft.

11. The transfer instrument according to claim 6, further comprising:
a second support portion having a sheet shape and including a second support surface configured to hold the medical sheet is provided to the distal portion of the second shaft; and
the second support portion is relatively movable with respect to the first support portion between a retracted position where the second support portion overlaps with the support surface and an advanced position where the second support portion is located in the distal end direction relative to the support surface.

12. The transfer instrument according to claim 11, wherein
at the first position, the second support portion is retracted inside the outer cylinder while being curved and deformed together with the first support portion.

13. A transfer instrument for transferring a medical sheet to a treatment site of a living body, the transfer instrument comprising:
an outer cylinder;
a first carrier member including a first shaft disposed inside the outer cylinder to be movable along an axial direction of the outer cylinder, and a first support portion having a sheet shape, disposed at a distal portion of the first shaft, and including a first support surface;
a second carrier member including a second shaft extending along the first shaft and provided movably along the first shaft, the second carrier member including a second support portion on a distal portion of the second shaft and having a sheet shape and including a second support surface, the second support portion is relatively movable with respect to the first support portion between a retracted position where the second support portion overlaps with the first support surface and an advanced position where the second support portion is located in the distal end direction relative to the first support surface;
wherein the first support portion is retracted into the outer cylinder in a state of being curved and deformed at a first position where the first and second shafts are moved in a proximal end direction with respect to the outer cylinder such that the first support portion is retracted into the outer cylinder, and the first support portion is unfolded by being exposed from the outer cylinder in the distal end direction at a second position where the first and second shafts are moved in the distal end direction with respect to the outer cylinder such that the first support portion protrudes from a distal-end opening of the outer cylinder;
a movement regulation portion that regulates relative movement of the first shaft and the second shaft in the axial direction between from the first position to the second position;
a movement range regulation portion that defines a distal end position of a relative movement range of the first shaft in the axial direction with respect to the outer cylinder; and
an engagement portion that is provided to the second carrier member and is engaged with the first carrier member to move the first carrier member in the proximal end direction when the second carrier member moves from the second position toward the first position.

14. The transfer instrument according to claim 13, wherein
the movement range regulation portion includes a coupling member having a string shape or wire shape and connected to the outer cylinder and the first carrier member; and
one end of the coupling member is connected to a proximal portion of the outer cylinder, and another end of the coupling member is connected to a proximal portion of the first shaft.

15. The transfer instrument according to claim 13, wherein
the movement range regulation portion includes a notch formed in one of the outer cylinder or the first shaft along the axial direction, and a projection that is formed on another one of the outer cylinder or the first shaft and slides along the notch.

16. The transfer instrument according to claim 13, wherein
the movement regulation portion comes into contact with the first support portion and a distal portion of the second shaft between from the first position to the second position, to regulate relative movement in the axial direction between the first shaft and the second shaft.

17. The transfer instrument according to claim 16, wherein
the movement regulation portion is a pressure-application portion configured by the distal portion of the second shaft; and
relative displacement between the first support portion and the second shaft is restricted by the pressure-application portion pressing the first support portion against an inner surface of the outer cylinder in a state where the first support portion is retracted in the outer cylinder.

18. The transfer instrument according to claim 16, wherein
the movement regulation portion includes a first fitting portion provided to the first support portion, and a second fitting portion provided to the distal portion of the second shaft;
relative displacement between the first support portion and the second shaft is restricted by fitting between the first fitting portion and the second fitting portion in a state where the first support portion is retracted in the outer cylinder; and
fitting between the first fitting portion and the second fitting portion is released by the first support portion being unfolded at the second position.

19. The transfer instrument according to claim 16, wherein
the movement regulation portion includes a first engagement portion provided on a front surface of the first support portion including the first support surface, and a second engagement portion formed at the distal portion of the second shaft;
in a state where the first support portion is retracted in the outer cylinder, relative displacement between the first support portion and the second shaft is restricted by the first engagement portion and the second engagement portion facing each other in the axial direction of the second shaft and the first engagement portion and the second engagement portion being engaged with each other in the axial direction; and
engagement between the first engagement portion and the second engagement portion is released without the first engagement portion and the second engagement portion facing each other in the axial direction, by the first support portion being unfolded at the second position.

20. A method of transferring a medical sheet to a treatment site of a living body, the method comprising:
placing a transfer instrument for transferring the medical sheet to the treatment site of the living body in an initial state, the transfer instrument including an outer cylinder, a first carrier member including a first shaft disposed inside the outer cylinder to be movable along an axial direction of the outer cylinder, and a first support portion having a sheet shape, disposed at a distal portion of the first shaft, and including a first support surface, a second carrier member including a second shaft extending along the first shaft and provided movably along the first shaft, the second carrier member including a second support portion on a distal portion of the second shaft and having a sheet shape and including a second support surface, the second support portion is relatively movable with respect to the first support portion between a retracted position where the second support portion overlaps with the first support surface and an advanced position where the second support portion is located in the distal end direction relative to the first support surface, wherein the first support portion is retracted into the outer cylinder in a state of being curved and deformed at a first position where the first and second shafts are moved in a proximal end direction with respect to the outer cylinder such that the first support portion is retracted into the outer cylinder, and the first support portion is unfolded by being exposed from the outer cylinder in the distal end direction at a second position where the first and second shafts are moved in the distal end direction with respect to the outer cylinder such that the first support portion protrudes from a distal-end opening of the outer cylinder, a movement regulation portion that regulates relative movement of the first shaft and the second shaft in the axial direction between from the first position to the second position, a movement range regulation portion that defines a distal end position of a relative movement range of the first shaft in the axial direction with respect to the outer cylinder, and an engagement portion that is provided to the second carrier member and is engaged with the first carrier member to move the first carrier member in the proximal end direction when the second carrier member moves from the second position toward the first position;
placing the medical sheet on the second support surface;
retracting the medical sheet into the outer cylinder together with the first support portion and the second support portion to a retracted position;
inserting the transfer instrument inserted into the living body and positioning a distal end of the transfer instrument near the treatment site of the living body;
moving the first shaft and the second shaft in a distal end direction relative to the outer cylinder via the movement regulation portion, and unfolding the first support portion, the second support portion and the medical sheet; and
moving the second carrier member in the distal end direction relative to the first carrier member so that the second support portion on which the medical sheet is placed is moved from the retracted position to an advanced position, and wherein the second support portion protrudes in the distal end direction relative to the distal end of the first support portion to move the medical sheet to above the treatment site of the living body and bringing an overhanging portion of the medical sheet into contact with the treatment site of the living body.
